(19) United States
(12) Patent Application Publication
(10) Pub. No.: US 2009/0130035 A1
(43) Pub. Date: May 21, 2009

(54) STABILIZED PREPARATIONS COMPRISING PHENOLIC COMPOUNDS AND BENZOPHENONES

(75) Inventors: Sabine Lange, Holzminden (DE); Gerhard Schmaus, Hoxter (DE); Gabriele Vielhaber, Holzminden (DE); Karin Schaper, Linnenkamp (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzminden (DE)

(21) Appl. No.: 12/159,866

(22) PCT Filed: Jan. 5, 2007
(86) PCT No.: PCT/EP07/50123

§ 371 Date: Oct. 20, 2008

Related U.S. Application Data
(60) Provisional application No. 60/756,205, filed on Jan. 5, 2006.
(60) Provisional application No. 60/804,319, filed on Jun. 9, 2006.

(51) Int. Cl. A61K 8/35; A61K 31/12; C07C 49/215

(52) U.S. Cl. 424/59; 514/683; 424/63; 568/309

(57) ABSTRACT

The present invention relates primarily to a cosmetic and/or pharmaceutical preparation comprising of consisting of one or more phenolic compounds of the formula (I)

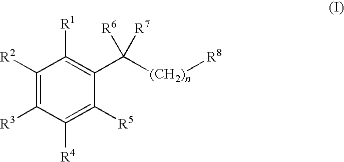

where for formula (I) it is the case that:
R¹, R², R³, R⁴ and R⁵ are independently of one another hydrogen, halogen, an OH group, methyl or a linear or branched alkyl having 2, 3, 4 or 5 C atoms, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl
   and one or more compounds of the formula (II)

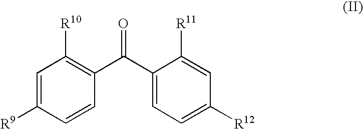

where for formula (II) it is the case that:
R⁹, R¹⁰, R¹¹ and R¹² independently of one another are hydrogen, methyl, an OH group or a group —O—R¹³ and
R¹³ is methyl or a linear or branched alkyl having 2, 3, 4, 5, 6, 7, 8, 9 or 10 C atoms,
   and to uses of compounds of the formula (II) for stabilizing compounds of the formula (I), and also to methods of stabilizing compounds of the formula (I).

### CROSS REFERENCE TO RELATED APPLICATIONS

The present application is a National Phase filing of PCT/EP2007/050123, which was filed on Jan. 5, 2007, and claims the benefit of U.S. Provisional Patent Application 60/604,319, filed on Jun. 9, 2006, and of U.S. Provisional Patent Application 60/756,205, filed on Jan. 5, 2006, the disclosures of which are incorporated by reference herein.

The present invention relates primarily to a cosmetic and/or pharmaceutical preparation comprising or consisting of one or more compounds of the formula (I)

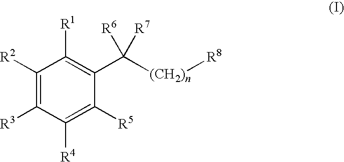

where for formula (I) it is the case that:
R¹, R², R³, R⁴ and R⁵ are independently of one another hydrogen, halogen, an OH group, methyl or a linear or branched alkyl having 2, 3, 4 or 5 C atoms, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl
   and
   one or more compounds of the formula (II)

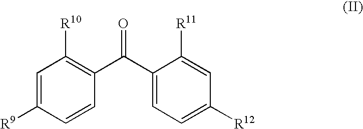

where for formula (II) it is the case that:
R⁹, R¹⁰, R¹¹ and R¹² independently of one another are hydrogen, methyl, an OH group or a group —O—R¹³ and
R¹³ is methyl or a linear or branched alkyl having 2, 3, 4, 5, 6, 7, 8, 9 or 10 C atoms,
   and to uses of compounds of the formula (II) for stabilizing compounds of the formula (I), and also to methods of stabilizing compounds of the formula (I).

In the field of the cosmetics industry there is an increasing demand for compositions for lightening skin and hair and also for compositions for combating age spots. Cosmetics for skin and hair lightening and for combating age spots play a large part in particular in Asian countries with a dark-skinned and dark-haired population, although in central Europe and in the USA, as well, compositions for cosmetic treatments of this kind are also becoming increasingly more important.

The skin and hair colour of people is determined substantially via the melanocytes count, via the melanin concentration and via the intensity of melanin biosynthesis with the skin and hair colour being affected significantly on the one hand by intrinsic factors such as the genetic make-up of an individual and on the other hand by extrinsic factors such as, in particular, the intensity and frequency of UV exposure.

Active skin- and hair-lightening compounds typically intervene in melanin metabolism and/or catabolism. The melanin pigments, which are generally brown to black in colour, are formed in the melanocytes of the skin, are transferred to the keratinocytes, and give rise to the coloration of the skin or hair. The brown-black eumelanins are formed in mammals principally from hydroxy-substituted aromatic amino acids such as L-tyrosine and L-DOPA, and the yellow to red phaeomelanins are formed additionally from sulphur-containing molecules (Cosmetics & Toiletries 1996, 111 (5), 43-51). Starting from L-tyrosine, the copper-containing key enzyme tyrosinase forms L-3,4-dihydroxyphenylalanine (L-DOPA), which is in turn converted into dopachrome by tyrosinase. Dopachrome is oxidized to melanin via a number of steps, which are catalysed by different enzymes.

In the search for new agents with a skin- and hair-lightening effect and/or an effect against age spots, accordingly, the aim is, very generally, to find substances which at a very low concentration inhibit the enzyme tyrosinase, a further factor to be borne in mind being that these substances, used in cosmetic and/or pharmaceutical products, must not only be highly active in very low concentrations but should also be
toxicologically unobjectionable,
well tolerated by the skin,
stable to temperature, pH and light (particularly in the typical cosmetic and/or pharmaceutical preparations),
preferably odourless, and
inexpensive to prepare (i.e. preparable using standard processes and/or from standard precursors).

In particular, various compounds of the formula (I), described for example in US 2003/0180234, US 2003/0185773, JP 2000327557, JP 2001010925, U.S. Pat. No. 4,959,393, EP 1 134 207, U.S. Pat. No. 6,132,740, WO 2000/56702 or U.S. Pat. No. 6,869,598, and also, especially, resorcinol derivatives described in DE 103 24 567, DE 103 24 566, WO 2004/105736 and U.S. Pat. No. 4,959,393, meet the majority of the aforementioned product requirements in an ideal way. In the case of the compounds described for example in WO 2004/105736 and U.S. Pat. No. 4,959,393, the skin- and hair-lightening and age-spot-reducing activity of substances of the formula (I) derives from the inhibition of tyrosinase, a key enzyme in melanin formation. This has been clearly demonstrated by corresponding in vitro experiments, including enzyme assays with fungal tyrosinase and also cell biology studies on B16 mouse melanoma cells.

The search for suitable (active) substances possessing one or more of the aforementioned properties to an adequate extent is made more difficult for the skilled person by the fact that there is no clear dependency between the chemical structure of a substance, on the one hand, and its biological activity and its stability, on the other. Moreover, there is no predictable connection between the skin- and hair-lightening effect, the toxicological unobjectionability, the skin tolerability and/or the stability of potential active compounds.

One particularly important prerequisite for the use of such an active substance in practice is its stability per se and in particular its stability in cosmetic and/or pharmaceutical preparations, which can be adversely affected by (solar and/or UV) light, under the influence of certain temperature or pH conditions, and also by chemical substances which are typically employed as accompanying substances in cosmetics or pharmaceutical preparations. Thus on the one hand it may be difficult to guarantee quantitatively consistent availability of the active substances, not least over a prolonged period of time, and on the other hand it may be difficult to prevent unwanted discolorations of the cosmetic and/or pharmaceutical preparations, which are usually preparations that can be applied topically.

It is well known that phenolic compounds, and here especially phenolic compounds having two or more OH groups, are unstable and have a tendency, for example, under the influence of light, under certain pH conditions, and in the presence of catalytic amounts of, for example, divalent metal ions, to undergo rearrangement reactions or degradation reactions, most of which are associated with a loss of substance and equally in many cases with a yellow or even brown discoloration of the substances themselves or of the cosmetic and/or pharmaceutical preparations which comprise phenolic compounds.

It is an object of the present invention, then, to allow phenolic compounds of the formula (I) to be formulated with long-term stability in respect of degradation and discoloration in cosmetic and/or pharmaceutical preparations. This object is achieved in accordance with the invention through a cosmetic and/or pharmaceutical preparation according to claim 1.

Surprisingly it has been found that in cosmetic and/or pharmaceutical preparations of the invention, comprising or consisting of
a) a tyrosinase-inhibiting amount of one or more compounds of the formula (I):

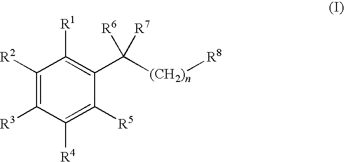

where for formula (I) it is the case that:
R¹, R², R³, R⁴ and R⁵ are independently of one another hydrogen, halogen, an OH group, methyl or a linear or branched alkyl having 2, 3, 4 or C atoms, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl
and
b) one or more compounds of the formula (II)

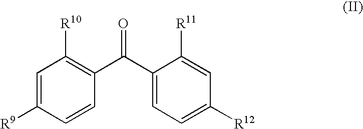

where for formula (II) it is the case that:
R⁹, R¹⁰, R¹¹ and R¹² independently of one another are hydrogen, methyl, an OH group or a group —O—R¹³
and
R¹³ is methyl or a linear or branched alkyl having 2, 3, 4, 5, 6, 7, 8, 9 or 10 C atoms,
   the compounds of the formula (II) are able to improve the stability of phenolic compounds of the formula (I) in cosmetic and/or pharmaceutical preparations to a high degree.

In particular the compounds of the formula (II) are able in this context to prevent or retard the discoloration of phenolic compounds of the formula (I) that is caused by sunlight or other light. Both prevention and retardation are important in particular in cosmetic preparations. In accordance with the invention, therefore, compounds of the formula (II) are used as UV filters for stabilizing the phenolic compounds of the formula (I), by employing one or more UV filters of the formula (II) in an amount sufficient for stabilizing the phenolic compounds of the formula (I) in a cosmetic and/or pharmaceutical preparation of the invention.

Preferred embodiments of the inventively preferred preparations and their uses are described below and also in the examples and the claims.

Preference is given to those preparations of the invention where for one or more compounds of the formula (I) it is the case that:
R¹, R², R³, R⁴ and R⁵ are independently of one another hydrogen or an OH group, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl.

Preference is given in particular to those preparations of the invention where for one or more compounds of the formula (I) it is the case that:
R¹, R², R³, R⁴ and R⁵ independently of one another are hydrogen, an OH group, halogen, methyl or a linear or branched alkyl having 2, 3 or 4 C atoms, of which two radicals from R¹, R², R³, R⁴ and R⁵ are hydrogen and two radicals are an OH group,
R⁶ is hydrogen, methyl or linear or branched alkyl having 2, 3, 4 or 5 C atoms,
R⁷ is methyl or linear or branched alkyl having 2, 3, 4 or 5 C atoms,
R⁸ is a phenyl
and
n is 0.

Preferred compounds of the formula (I) are those for which

R⁶ is hydrogen or methyl,
R⁷ is hydrogen, methyl or ethyl,
n is an integer from 0 to 8 and
R⁸ is methyl, cyclopentyl, cyclohexyl or phenyl.

Particularly preferred phenolic compounds of the formula (I) are those of the formulae (IB) and (IC), in each of which R¹ and R⁷ have the preferred definition specified above, and in formula (IC) n is an integer from 0 to 4:

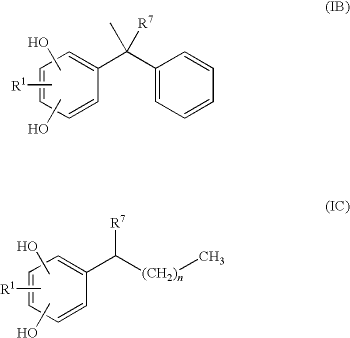

The OH group and R¹ as substituents may in each case (as depicted by drawing) occupy any desired position on the aromatic ring.

Very particular preference is given to the following phenolic compounds of the formula (I):
4-butylresorcinol (CARN: 18979-61-8)
and
in particular to styrylresorcinol described in more detail below (formula (IA), CARN: 85-27-8; 4-(1-phenylethyl)-1,3-dihydroxybenzene):

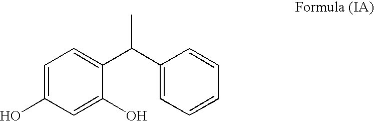

Preference is further given to a preparation of the invention comprising
(i) styrylresorcinol as compound of the formula (I)
and
(ii) 2-hydroxy-4-methoxybenzophenone as compound of the formula (II)
   in a weight ratio of 5:1. A preparation of this kind has already been described in U.S. Prov. 60/756,205 (Symrise GmbH & Co. KG).

Preferred compounds of the formula (II) are those for which:

R⁹, R¹⁰, R¹¹ and R¹² are independently of one another hydrogen, methyl, methoxy or n-octoxy.

Particularly preferred compounds of the formula (II) are for example:
2,2′-dihydroxy-4,4′-dimethoxybenzophenone,
2-hydroxy-4-n-octoxybenzophenone,
2-hydroxy-4-methoxy-4′-methylbenzophenone,
dihydroxy-4-methoxybenzophenone,
2,4-dihydroxybenzophenone (INCI: Benzophenone-1; formula (IIA))

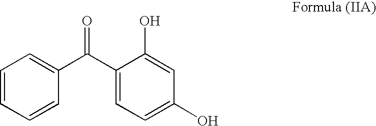

2,2′,4,4′-tetrahydroxybenzophenone (INCI: Benzophenone-2; formula (IIB))

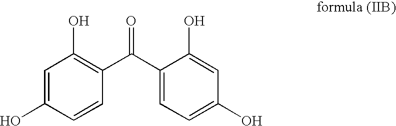

and
2-hydroxy-4-methoxybenzophenone (INCI: Benzophenone-3; formula (IIC))

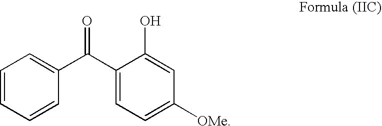

Very particularly preferred compounds of the formula (II) are:
2,4-dihydroxybenzophenone (INCI: Benzophenone-1; formula (IIA)),
2,2′,4,4′-tetrahydroxybenzophenone (INCI: Benzophenone-2; formula (IIB))
and
2-hydroxy-4-methoxybenzophenone (INCI: Benzophenone-3; formula (IIC)).

These preferred compounds can be formulated without decomposition and with colour stability in cosmetic and pharmaceutical preparations, with the aid in particular of the inventive use of the compound of the formula (II), and display an outstanding skin- and hair-lightening effect and also an outstanding age-spot-reducing effect even over a long time period.

One preferred embodiment of the present invention has been possible to show by means of further comparative stability tests with preparations that also, by adjusting the pH to values less than or equal to 5.5 and preferably less than or equal to 4.5 it is possible to produce additional protection of phenolic compounds of the formula (I) against degradation and associated discoloration.

Further comparative stability tests with preparations have shown that by adding metal chelators it is also possible to produce additional protection of phenolic compounds of the formula (I) against degradation and associated discoloration.

Metal chelators which can be used with particular advantage in this context are, in particular, α-hydroxy fatty acids, phytic acid, lactoferrin, α-hydroxy acids and their salts, including citric acid, lactic acid and malic acid, and also galactaric acid, galacturonic acid, gluconic acid, glucuronic acid, ribonic acid and salts thereof, humic acids, gallic acids, bile extracts, bilirubin, biliverdin, and EDTA, EGTA and derivatives thereof.

Further in-house comparative stability tests have shown that preparations to which with particular preference the photoprotective filters of the invention have been added, but to which, additionally, metal chelators have been added and which, moreover, have been adjusted optimally in terms of their pH, allow very particular protection of phenolic compounds of the formula (I) against degradation and associated discoloration.

A significantly reduced degradation and a significant reduction of discolorations of phenols of the formula (I) was achieved in this context in particular through the preparation of the invention, in respect of which photoprotective filters of the formula (II) shown below were added to the cosmetic or pharmaceutical preparations in an appropriate mixing ratio, it being possible to combine this method of the invention, preferably, additionally with further methods of improving stability, such as the addition of disodium ethylenediaminetetraacetate (INCI: Disodium EDTA) or other metal chelators and/or the adjustment of the pH to values less than or equal to 5.5 and preferably less than or equal to 4.5.

When phenolic compounds of the formula (I) are used for tyrosinase inhibition in cosmetic and/or pharmaceutical preparations, these compounds are used primarily for cosmetological reasons, though in exceptional cases they may also possess a therapeutic character.

The concentration of the amount of active compound for application daily, for example, is different and depends on the physiological condition of the subject and also on parameters specific to the individual, such as age or bodyweight. Compounds of the formula (I), alone, as mixtures or else in combination with further tyrosinase-inhibiting substances, can be employed in the preparations of the invention.

The compounds of the formula (I) can be incorporated without difficulties, using the stabilization methods of the invention, into all conceivable cosmetic and/or pharmaceutical preparations. In this context it is also possible, and advantageous in certain cases, to combine the compounds of the formula (I) in the cosmetic and/or pharmaceutical preparations with further active compounds, such as with other compounds having a skin- and hair-lightening action or compounds which are active against age spots. The cosmetic and/or dermatological/keratological preparations of the invention may in this context have the typical composition and may serve for the treatment of the skin and/or hair as a dermatological or keratological treatment or as a treatment in the sense of cosmetic care treatment. They can alternatively be employed in decorative cosmetics.

A further advantageous preparation of the invention has a water content of 25% to 95% by weight, preferably of 40% to 90% by weight, based in each case on the total weight of the preparation.

Moreover, a preparation of the invention having an oil phase weight fraction of 0.05% to 12% by weight is advantageous.

For the purposes of this invention, depending on the weight fraction of the oil phase in the preparation of the invention, an oil phase can be present in emulsified form or in solution in the preparation.

The oil phase in a preparation of the invention can also be designated as a component of that preparation. The hydrophilic fraction in such a preparation of the invention can be designated as a hydrophilic component of that preparation.

The oil phase itself may be composed in turn of one or more oil components, which are described in more detail later on (see below).

Surprisingly, in-house comparative human in vivo studies with preparations having different oil phase contents have shown that, in particular, preparations having a high fraction of hydrophilic components and a sharply reduced fraction of the oil phase achieve these objectives to particularly good effect and can therefore be employed with particular preference as transport systems for tyrosinase inhibitors of the formula 1.

A significantly greater skin- and hair-lightening and age-spot-reducing activity and a significantly higher bioavailability have been found in particular for a preparation of the invention in which the fraction of the oil phase is in the range from 0.05% to 12% by weight, based on the total weight of the preparation.

Preparations of the invention may be present preferably in the form of an O/W emulsion.

Preparations of the invention advantageously comprise one or more further UV filters and/or one or more further tyrosinase inhibitors.

Preferred preparations of the invention comprise a total amount of UV filters and/or inorganic pigments such that the preparation of the invention has a sun protection factor of greater than or equal to 2, preferably greater than or equal to 5.

Preference is given, moreover, to preparations of the invention which comprise a further active skin- and/or hair-lightening compound, preferably in an active skin- and/or hair-lightening amount.

Preference extends to preparations of the invention which comprise an active cooling compound in an amount sufficient to achieve a skin-cooling effect.

Preference is likewise given to preparations of the invention which comprise one or more compounds for the care and/or cleansing of (a) skin and/or (b) hair.

Preference is given, furthermore, to preparations of the invention which comprise a sensorially active amount of one or more odoriferous substances.

The significantly best improvement in stability of the compounds of the formula (I) has been found for the mixtures of the invention in which the fraction of photoprotective filters of the general formula (II) and especially of photoprotective filters having the formulae (IIA), (IIB) and (IIC) in the ready-to-use cosmetic and/or pharmaceutical preparations is in a concentration range from 0.05% to 12% by weight, preferably from 0.1% to 10% by weight and with particular preference from 0.5% to 8% by weight, based on the total weight of the preparation.

For preparations of the invention comprising at least one of the said photoprotective filters in the preferred quantitative ranges, in particular, a very good long-term stability and/or a very good colour stability of the phenolic compound of the formula (I) have/has been found experimentally.

In the context of this specification, long-term stability means that for at least 6 months, preferably at least 12 months (in each case at 25° C.), the rate of degradation of the compounds of the formula (I) is generally less than or equal to 10% by weight, preferably less than or equal to 5% by weight, based on the total weight of the compounds of the formula (I) introduced originally into a preparation of the invention.

Furthermore, it has proved to be particularly advantageous to predissolve the compounds of the formula (I) in an oil phase (one or more oil components, preferably those specified below) to which the photoprotective filters of the formula (II) have been added in a defined mixing ratio. The oil phase thus stabilized, comprising one or more compounds of the formula (I) (in the text below, a mixture of this kind is referred to as a “premix”), may be mixed, in a second, subsequent step, for producing a ready-to-use cosmetic or pharmaceutical preparation, with the other ingredients of the cosmetic or pharmaceutical preparation.

A further aspect of the present invention relates to a premix of the invention comprising or consisting of:
a) one or more compounds of the formula (I):

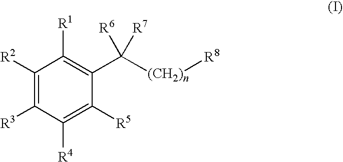

where for formula (I) it is the case that:
R¹, R², R³, R⁴ and R⁵ are independently of one another hydrogen, halogen, an OH group, methyl or a linear or branched alkyl having 2, 3, 4 or 5 C atoms, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl
   and
b) one or more compounds of the formula (II):

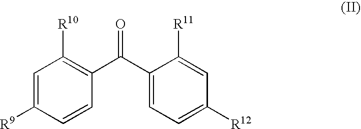

where for formula (II) it is the case that:
R⁹, R¹⁰, R¹¹ and R¹² independently of one another are hydrogen, methyl, an OH group or a group —O—R¹³
and
R¹³ is methyl or a linear or branched alkyl having 2, 3, 4, 5, 6, 7, 8, 9 or 10 C atoms,
   and
c) one or more oil components, the total amount of the oil component in the premix being 45% by weight or more, preferably 60% by weight or more, based on the total amount of the premix.

For the premix it is likewise possible to use the preferred compounds of the formula (I) and (II).

A premix is preferably water-free. If desired, a premix may comprise further components, such as metal chelators, for example.

In one preferred embodiment the premix of the invention comprises one or more photoprotective filters from the group of the compounds of the formulae (IIA), (IIB) and (IIC) in quantitative proportions of 0.1% to 10% by weight and preferably of 0.5% to 2% by weight, based on the weight of the total mixture.

The total amount of the phenolic compounds of the formula (I) that is used with preference in relation to the total amount of the photoprotective filters of the formula (II) depends on the nature of the preparation (formulation) and advantageously exhibits an amount sufficient to stabilize one or more compounds of the formula (I) in the premix.

In a premix (as described above) the weight-based ratio of the phenolic compounds of the formula (I) to the total amount of the photoprotective filters of the formula (II) is greater than 5:1 and is situated preferably in the range from 50:1 to 6:1, preferably in the range from 30:1 to 8:1.

In a ready-to-use cosmetic or pharmaceutical preparation of the invention the weight-based ratio of the phenolic compounds of the formula (I) to the total amount of the photoprotective filters of the formula (II) is less than 5:1 and is situated preferably in the range from 4:1 to 1:20, preferably in the range from 2:1 to 1:12.

A suitable oil phase or suitable oil component in the sense of the present invention encompasses the following groups of substances:
(i) linear or branched saturated paraffins (mineral oils) having 15 or more C atoms, in particular having 18 to 45 C atoms;
(ii) esters having 12 or more C atoms of linear or branched fatty acids having 6 to 30 C atoms and linear or branched, saturated or unsaturated mono-, di- or triols having 3 to 30 C atoms, these esters having no free hydroxyl groups;
(iii) esters of benzoic acid and linear or branched, saturated or unsaturated monoalkanols having 8 to 20 C atoms;
(iv) monoesters or diesters of alcohols having 3 to 30 C atoms and naphthalene-monocarboxylic or -dicarboxylic acids; especially naphthalenemonocarboxylic acid C₆-C₁₈ esters and naphthalenedicarboxylic acid di-C₆-C₁₈ esters;
(v) linear or branched, saturated or unsaturated di-C₆-C₁₈-alkyl ethers;
(vi) silicone oils;
(vii) 2-alkyl-1-alkanols of the formula (III)

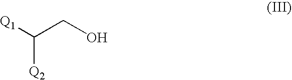

where
Q₁ is a linear or branched alkyl radical having 6 to 24 C atoms and
Q₂ is a linear or branched alkyl radical having 4 to 16 C atoms.

An oil phase or oil component in the narrower (and preferred) sense of the present invention, i.e. of the inventively limited substances or substances present only in a minor fraction, encompasses the following groups of substances:
(i) linear or branched, saturated paraffins having 20 to 32 C atoms;
(ii) esters having at least 14 C atoms of linear or branched, saturated fatty acids having 8 to 24 C atoms and linear or branched, saturated or unsaturated mono-, di- or triols having 3 to 24 C atoms, these esters containing no free hydroxyl groups;
(iii) esters of benzoic acid and linear or branched, saturated monoalkanols having 10 to 18 C atoms;
(iv) 2,6-naphthalenedicarboxylic acid di-C6-C12 esters;
(v) linear or branched, saturated di-C6-C18-alkyl ethers, especially (straight-chain) di-C6-C12-alkyl ethers;
(vi) silicone oils from the group of the cyclotrisiloxanes, cyclopentasiloxanes, dimethylpolysiloxanes, diethylpolysiloxanes, methylphenylpolysiloxanes, diphenylpolysiloxanes and hybrid forms thereof;
(vii) 2-alkyl-1-alkanols having 12 to 32 C atoms of the formula (III)
   where
   Q₁ is a (preferably linear) alkyl radical having 6 to 18 C atoms and
   Q₂ is a (preferably linear) alkyl radical having 4 to 16 C atoms.

An oil phase in the narrowest (and most preferred) sense of the present invention encompasses the following groups of substances:
(i) linear or branched, saturated paraffins having 20 to 32 C atoms such as isoeicosane or squalane;
(ii) esters having at least 16 C atoms of linear or branched, saturated fatty acids having 8 to 18 C atoms and linear or branched, saturated mono-, di- or triols having 3 to 18 C atoms, these esters containing no free hydroxyl groups;
(iii) esters of benzoic acid and linear or branched, saturated monoalkanols having 12 to 15 C atoms, especially C₁₂₋₁₅-alkyl benzoates;
(iv) 2,6-naphthalenedicarboxylic acid di-C6-C10 esters, especially diethylhexyl 2,6-naphthalenedicarboxylate;
(v) straight-chain di-C₆-C₁₀-alkyl ethers; especially di-n-octyl ether (dicaprylyl ether);
(vi) silicone oils from the group undecamethylcyclotrisiloxane, cyclomethicone, decamethylcyclopentasiloxane, dimethylpolysiloxanes, diethylpolysiloxanes, methylphenylpolysiloxanes and diphenylpolysiloxanes;
(vii) 2-alkyl-1-alkanols having 12 to 32 C atoms of the formula (III)
   where
   Q₁ is a (preferably linear) alkyl radical having 6 to 18 C atoms and
   Q₂ is a (preferably linear) alkyl radical having 4 to 16 C atoms.

Particularly preferred components of type (i) in the oil phase are as follows: isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, 2-ethylhexyl isostearate, isotridecyl isononanoate, 2-ethylhexyl cocoate, caprylic/capric triglyceride, and also synthetic, semisynthetic and natural mixtures of such esters, e.g. jojoba oil.

Fatty acid triglycerides (oil components of type (i) in the oil phase) may also be in the form of, or in the form of a constituent of, synthetic, semisynthetic and/or natural oils, examples being olive oil, sunflower oil, soya oil, peanut oil, rapeseed oil, almond oil, palm oil, coconut oil, palm kernel oil and mixtures thereof.

Particularly preferred oil components of type (vii) in the oil phase are as follows: 2-butyl-1-octanol, 2-hexyl-1-decanol, 2-octyl-1-dodecanol, 2-decyltetradecanol, 2-dodecyl-1-hexadecanol and 2-tetradecyl-1-octadecanol.

Particularly preferred oil components in the oil phase are mixtures comprising C₁₂-C₁₅-alkyl benzoate and 2-ethylhexyl isostearate, mixtures comprising C₁₂-C₁₅-alkyl benzoate and isotridecyl isononanoate, mixtures comprising C₁₂-C₁₅-alkyl benzoate, 2-ethylhexyl isostearate and isotridecyl isononanoate, mixtures comprising cyclomethicone and isotridecyl isononanoate, and mixtures comprising cyclomethicone and 2-ethylhexyl isostearate.

A further aspect of the present invention relates to a method of the invention of stabilizing compounds of the formula (I) by preparing a mixture comprising or consisting of:
a) one or more compounds of the formula (I)

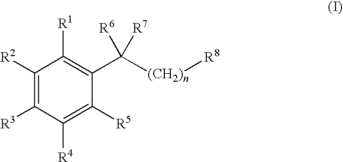

where for formula (I) it is the case that:
R¹, R², R³, R⁴ and R⁵ are independently of one another hydrogen, halogen, an OH group, methyl or a linear or branched alkyl having 2, 3, 4 or 5 C atoms, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl
   and
b) one or more compounds of the formula (II)

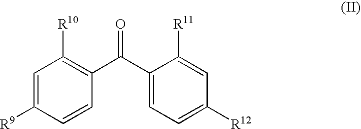

where for formula (II) it is the case that:
R⁹, R¹⁰, R¹¹ and R¹² independently of one another are hydrogen, methyl, an OH group or a group —O—R¹³
and
R¹³ is methyl or a linear or branched alkyl having 2, 3, 4, 5, 6, 7, 8, 9 or 10 C atoms
   and
c) if desired, one or more oil components.

For this method of the invention it is likewise possible to use the preferred compounds of the formula (I) and (II).

One of the ways in which stabilization of the compounds of the formula (I) can be determined is via the long-term stability and/or the improvement in the colour stability of the compounds of the formula (I).

In one preferred embodiment a method of the invention of this kind has as the resulting mixture a preparation of the invention or a premix of the invention.

In one preferred method of the invention, where the resulting mixture is a preparation of the invention, the method comprises or consists of the following steps:
a) providing one or more compounds of the formula (I),
b) providing a mixture comprising one or more compounds of the formula (II) and one or more oil components, the total amount of the oil component being 45% by weight or more, based on the mixture,
c) dissolving one or more compounds of the formula (I) from step a) in the mixture from step b)
and subsequently
d) mixing the resulting premix from step c) with further ingredients of a cosmetic and/or pharmaceutical preparation.

A further aspect of the present invention relates to a method of lightening skin and/or hair and/or of reducing age spots, comprising or consisting of the step of:
applying a preparation of the invention to skin and/or hair.

A further aspect of the present invention relates to the use of one or more compounds of the formula (II)

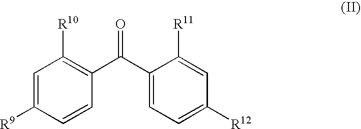

where for formula (II) it is the case that:
R⁹, R¹⁰, R¹¹ and R¹² are independently of one another hydrogen, methyl, an OH group or a group —O—R¹³
and
R¹³ is methyl or a linear or branched alkyl having 2, 3, 4, 5, 6, 7, 8, 9 or 10 C atoms,
for preparing a mixture comprising or consisting of one or more compounds of the formula (II)
and
one or more compounds of the formula (I)

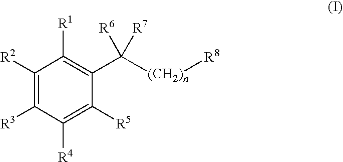

where for formula (I) it is the case that:
R¹, R², R³, R⁴ and R⁵ are independently of one another hydrogen, halogen, an OH group, methyl or a linear or branched alkyl having 2, 3, 4 or 5 C atoms, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl.

For this method of the invention it is likewise possible to use the preferred compounds of the formula (I) and (II).

A further aspect of the present invention relates to the use of one or more compounds of the formula (II)

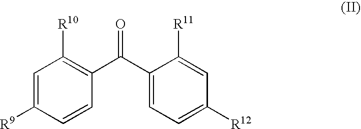

where for formula (II) it is the case that:
R⁹, R¹⁰, R¹¹ and R¹² are independently of one another hydrogen, methyl, an OH group or a group —O—R¹³
and
R¹³ is methyl or a linear or branched alkyl having 2, 3, 4, 5, 6, 7, 8, 9 or 10 C atoms,
for the long-term stabilization and/or colour stabilization of one or more compounds of the formula (I)

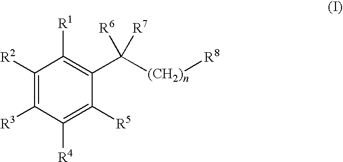

where for formula (I) it is the case that:
R¹, R², R³, R⁴ and R⁵ are independently of one another hydrogen, halogen, an OH group, methyl or a linear or branched alkyl having 2, 3, 4 or 5 C atoms, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl.

For this inventive use it is likewise possible to use the preferred compounds of the formula (I) and (II).

The preparations which are stabilized by means of the methods of the invention and comprise one or more compounds of the formula (I) and one or more compounds of formula (II) are preferably part of the following preparations or are present in one of the following forms:

Emulsion of the “oil-in-water” (O/W) type, PIT emulsion, Pickering emulsion, microemulsion, pencil, stick, spray, foam, impregnating solution, for cosmetic wipes for example, cleansing composition such as, for example, soap, syndet, liquid washing, showering and bathing product; skincare composition, cream, lotion, milk, emulsion foam, microemulsion, nanoemulsion, paste, gel (e.g. hydrogel or hydrodispersion gel), balsam, serum, roll-on, pump spray, aerosol (foaming, non-foaming or after-foaming), footcare composition (including keratolytics, deodorants), insect repellent, sunscreen, after-sun product, shaving composition, depilatory, haircare composition such as, for example, shampoo, 2-in-1 shampoo, anti-dandruff shampoo, baby shampoo, shampoo for dry scalp, shampoo concentrate, conditioner, hair tonic, hair lotion, hair rinse, styling cream, permanent-waving and setting composition, hair smoothing composition (straightening composition, relaxer), hair setting composition (spray), styling aid (e.g. gel), as a bleach-blonding composition, hair lightener, hair conditioner, hair mousse, hair tint, deodorant and/or antiperspirant; mouthwash and mouth spray, aftershave balm, pre- and aftershave lotion, eyecare, makeup, makeup remover, baby article, bath article (e.g. capsule), or mask. It is further advantageous to present the compounds of the formula (I) in encapsulated form, such as in liposomes or cellulose capsules, for example.

The cosmetic and/or pharmaceutical (therapeutic) preparations, especially topical preparations, stabilized by means of the methods of the invention, and especially skin-lightening and hair-lightening compositions, may comprise cosmetic and/or pharmaceutical auxiliaries and additives, such as are typically used in preparations of this kind, examples being sunscreen agents, preservatives, bactericides, fungicides, virucides, active cooling compounds, insect repellents (e.g. DEET, IR 3225, Dragorepel), plant extracts, active anti-inflammatory compounds, substances which accelerate wound healing (e.g. chitin or chitosan and its derivatives), film-forming substances (e.g. polyvinylpyrrolidones or chitosan or its derivatives), customary antioxidants, vitamins (e.g. vitamin C and derivatives, tocopherols and derivatives, vitamin A and derivatives), 2-hydroxycarboxylic acids (e.g. citric acid, malic acid, L-, D- or dl-lactic acid), skincare agents (e.g. cholesterol, ceramides, pseudoceramides), softening, moisturizing and/or humectant substances (especially glycerol, urea or 1,2-alkanediols such as 1,2-pentanediol, 1,2-hexanediol and/or 1,2-octanediol), saturated fatty acids, mono- or polyunsaturated fatty acids, alpha-hydroxy acids, polyhydroxy fatty acids or derivatives thereof (e.g. linoleic acid, alpha-linolenic acid, gamma-linolenic acid or arachidonic acid and their respective natural or synthetic esters), waxes or other typical constituents of a cosmetic or dermatological preparation, such as alcohols, polyols, polymers, foam stabilizers, electrolytes, organic solvents, silicone derivatives, active anti-dandruff compounds (e.g. climbazole, ketoconazole, piroctone oleamine, zinc pyrithione), haircare agents, perfume, foam preventatives, dyes, pigments which have a colouring action, thickeners, surface-active substances, surfactants, emulsifiers, plant parts and plant extracts (e.g. arnica, aloe, beard lichen, ivy, stinging nettle, ginseng, henna, camomile, marigold, rosemary, sage, blackberry, horsetail or thyme), royal jelly, propolis, proteins, protein hydrolysates, yeast extracts, hop extracts and wheat extracts, peptides or thymus extracts.

The particular amounts of cosmetic and/or pharmaceutical, especially dermatological, auxiliaries and additives, and also one or more odoriferous substances (perfumes), to be employed can easily be determined in accordance with the nature of the respective product by means of simple trialing by the skilled person.

The preparations of the invention which comprise phenolic compounds of the formula (I) may also comprise further active compounds having a skin-lightening effect. In accordance with the invention it is possible in this context to use all active skin-lightening compounds which are customary or suitable for cosmetic and/or pharmaceutical, especially dermatological, applications.

Advantageous active skin-lightening compounds in this respect are kojic acid (5-hydroxy-2-hydroxymethyl-4-pyranone), kojic acid derivatives such as kojic dipalmitate for example, arbutin, ascorbic acid, ascorbic acid derivatives, hydroquinone, hydroquinone derivatives, molecules containing sulphur, such as glutathione or cysteine for example, alpha-hydroxy acids (e.g. citric acid, lactic acid, malic acid) and their derivatives, N-acetyltyrosine and derivatives, undecenoylphenylalanine, gluconic acid, chromone derivatives such as aloesin, flavonoids, thymol derivatives, 1-aminoethylphosphinic acid, thiourea derivatives, ellagic acid, nicotinamide, zinc salts such as zinc chloride or zinc gluconate for example, thujaplicin and derivatives, triterpenes such as maslic acid, sterols such as ergosterol, benzofuranones such as senkyunolide, vinyl- and ethylguaiacol, dionic acids such as octodecenedionic acid and azelaic acid, nitrogen oxide synthesis inhibitors such as L-nitroarginine and its derivatives, 2,7-dinitroindazole or thiocitrulline, metal chelators (e.g. alpha-hydroxy fatty acids, palmitic acid, phytic acid, lactoferrin, humic acid, gallic acid, bile extracts, bilirubin, biliverdin), retinoids, soja milk, soya extract, serine protease inhibitors or lipoic acid or other synthetic or natural active compounds for skin and hair lightening, these compounds also being used in the form of an extract from plants, such as bearberry extract, rice extract, papaya extract, liquorice root extract or constituents concentrated from these, such as glabridin or licochalcone A, Artocarpus extract, extract from Rumex and Ramulus species, extracts from pine species (Pinus) and extracts from Vitis species or stilbene derivatives concentrated from these, extract from saxifraga, mulberry, Scutelleria and/or grapes.

For the use of the cosmetic and/or pharmaceutical, especially dermatologically, active preparations of the invention applied in sufficient amount to the skin and/or hair in the way which is typical for cosmetic and dermatological products. Particular advantages are offered in this context in particular by those cosmetic and dermatological preparations which as well as the photoprotective filters of the formula (II), used principally for stabilization with respect to degradation and discoloration, comprise one or more further sunscreen filters (UV absorbers, UV filters), as a result of which, on the one hand, the phenolic compounds of the formula (I) contained in the preparations are protected even more effectively with respect to degradation and colour changes, and, on the other hand, the preparations act equally as a hair- or skin-lightening and/or age-spot-reducing product and also as a sunscreen.

In one preferred embodiment cosmetic and/or pharmaceutical preparations of the invention comprise one or more further sunscreen filters (UV absorbers), preferably a total fraction of UV absorbers in the range from 0.1% to 30% by weight, more preferably in the range from 0.2% to 20% by weight, in particular 0.5% to 15% by weight, based on the total weight of the preparation.

For the purposes of stabilization the total amount of UV filters is preferably in the range from 0.01% to 10% by weight, in particular 0.05% to 5% by weight, based on the total weight of the preparation.

In a further preferred embodiment a cosmetic and/or pharmaceutical preparation of the invention, especially a dermatologically active preparation, comprises a total amount of UV filters and/or inorganic pigments such that the preparation of the invention has a sun protection factor of greater than or equal to 2 (preferably greater than or equal to 5). These sunscreens are suitable for protecting skin and hair.

In a further preferred embodiment a cosmetic and/or pharmaceutical preparation of the invention, especially a dermatologically active preparation, further comprises one or more sunscreen filters (UV absorbers), it being possible for these sunscreen filters to be present in various forms, of the kind typically used for sunscreen preparations, for example. Thus they may be for example in the form of an emulsion of the oil-in-water (O/W) type, a gel, a hydrodispersion, or else an aerosol.

Preference is given here to cosmetic and/or pharmaceutical preparations of the invention which comprise a photoprotective filter of the formula (II), with particular preference one or more photoprotective filters of the formulae (IIA), (IIB) and (IIC).

Advantageously it is possible for the preparations to comprise one or more photoprotective filters selected from the group of the compounds of the formula (II), but also UV-A filters or UV-B filters

and/or at least one further broadband filter
and/or
at least one inorganic pigment.

Further suitable photoprotective agents (UV absorbers) are, for example, organic UV absorbers from the class of 4-aminobenzoic acid and derivatives, salicylic acid derivatives, further benzophenone derivatives, further dibenzoylmethane derivatives, diphenylacrylates, 3-imidazol-4-ylacrylic acid and its esters, benzofuran derivatives, benzylidenemalonate derivatives, polymeric UV absorbers containing one or more organosilicon radicals, cinnamic acid derivatives, camphor derivatives, trianilino-s-triazine derivatives, 2-hydroxyphenylbenzotriazole derivatives, phenylbenzimidazolesulphonic acid derivatives and salts thereof, menthyl anthranilate, benzotriazole derivatives, indole derivatives.

The UV absorbers specified below, which can be used additionally for the purposes of the present invention, are preferred, but of course are not limiting.

Further preferred UV filters are as follows:

UV-B filters such as, for example:
p-aminobenzoic acid
ethyl p-aminobenzoate (25 mol) ethoxylated
2-ethylhexyl p-dimethylaminobenzoate
ethyl p-aminobenzoate (2 mol) N-propoxylated
glycerol p-aminobenzoate
homomethyl salicylate (homosalate) (Neo Heliopan®HMS)
2-ethylhexyl salicylate (Neo Heliopan®OS)
triethanolamine salicylate
4-isopropylbenzyl salicylate
menthyl anthranilate (Neo Heliopan®MA)
ethyl diisopropylcinnamate
2-ethylhexyl p-methoxycinnamate (Neo Heliopan®AV)
methyl diisopropylcinnamate
isoamyl p-methoxycinnamate (Neo Heliopan®E 1000)
p-methoxycinnamic acid diethanolamine salt
isopropyl p-methoxycinnamate
2-phenylbenzimidazolesulphonic acid and salts (Neo Heliopan®Hydro)
3-(4′-trimethylammonium)benzylidenebornan-2-one methyl sulphate
R-imidazole-4(5)-acrylic acid (urocanic acid)
3-(4′-sulpho)benzylidenebornan-2-one and salts
3-(4′-methylbenzylidene)-d,l-camphor (Neo Heliopan®MBC)
3-benzylidene-d,l-camphor
N-[(2 and 4)-[2-(oxoborn-3-ylidene)methyl]benzyl]acrylamide polymer
4,4′-[(6-[4-(1,1-dimethyl)aminocarbonyl)phenylamino]-1,3,5-triazine-2,4-diyl)diimino]bis(benzoic acid 2-ethylhexyl ester) (Uvasorb HEB)
benzylidenemalonate-polysiloxane (Parsol®SLX)
glyceryl ethylhexanoate dimethoxycinnamate
dipropylene glycol salicylate
tris(2-ethylhexyl) 4,4′,4″-(1,3,5-triazine-2,4,6-triyltriimino)tribenzoate (Uvinul®T150)

The following broadband filters are preferred, such as, for example:
2-ethylhexyl 2-cyano-3,3-diphenylacrylate (Neo Heliopan®303)
ethyl 2-cyano-3,3′-diphenylacrylate
2-hydroxy-4-methoxybenzophenone-5-sulphonic acid
sodium hydroxymethoxybenzophenonesulphonate
disodium 2,2′-dihydroxy-4,4′-dimethoxy-5,5′-disulphobenzophenone
phenol, -(2H-benzotriazol-2-yl-4-methyl-6-(2-methyl-3-(1,3,3,3-tetramethyl-1-(trimethylsilyl)oxy)disiloxyanyl)propyl), (Mexoryl®XL)
2,2′-methylenebis(6-(2H-benztriazol-2-yl)-4-1,1,3,3-tetramethylbutyl)-phenol), (Tinosorb®M)
2,4-bis[4-(2-ethylhexyloxy)-2-hydroxyphenyl]-1,3,5-triazine
2,4-bis[{(4-(2-ethylhexyloxy)-2-hydroxy}phenyl]-6-(4-methoxyphenyl)-1,3,5-triazine, (Tinosorb®S)
2,4-bis[{(4-(3-sulphonato)-2-hydroxypropyloxy)-2-hydroxy}phenyl]-6-(4-methoxyphenyl)-1,3,5-triazine sodium salt
2,4-bis[{(3-(2-propyloxy)-2-hydroxypropyloxy)-2-hydroxy}phenyl]-6-(4-methoxyphenyl)-1,3,5-triazine
2,4-bis[{4-(2-ethylhexyloxy)-2-hydroxy}phenyl]-6-[4-(2-methoxyethyl-carbonyl)phenylamino]-1,3,5-triazine
2,4-bis[{4-(3-(2-propyloxy)-2-hydroxypropyloxy)-2-hydroxy}phenyl]-6-[4-(2-ethylcarboxyl)phenylamino]-1,3,5-triazine
2,4-bis[{4-(2-ethylhexyloxy)-2-hydroxy}phenyl]-6-(1-methylpyrrol-2-yl)-1,3,5-triazine
2,4-bis[{4-tris(trimethylsiloxysilylpropyloxy)-2-hydroxy}phenyl]-6-(4-methoxyphenyl)-1,3,5-triazine
2,4-bis[{4-(2″-methylpropenyloxy)-2-hydroxy}phenyl]-6-(4-methoxyphenyl)-1,3,5-triazine
2,4-bis[{4-(1′,1′,1′,3′,5′,5′,5′-heptamethylsiloxy-2″-methylpropyloxy)-2-hydroxy}phenyl]-6-(4-methoxyphenyl)-1,3,5-triazine.

The following UV-A filters are preferred, such as, for example:
terephthalylidenedibornanesulphonic acid and salts (Mexoryl®SX)
4-t-butyl-4′-methoxydibenzoylmethane (avobenzone)/(Neo Heliopan®357)
phenylenebisbenzimidazyltetrasulphonic acid disodium salt (Neo Heliopan®AP)
2,2′-(1,4-phenylene)bis(1H-benzimidazole-4,6-disulphonic acid), monosodium salt
hexyl 2-(4-diethylamino-2-hydroxybenzoyl)benzoate (Uvinul® A Plus)
4-isopropyldibenzoylmethane
indanylidene compounds as per DE 100 55 940 (=WO 02/38537).

UV absorbers particularly suitable for combination here are as follows:
p-aminobenzoic acid
3-(4′-trimethylammonium)benzylidenebornan-2-one methyl sulphate
homomethyl salicylate (Neo Heliopan®HMS)
2-phenylbenzimidazolesulphonic acid (Neo Heliopan®Hydro)
terephthalylidenedibornanesulphonic acid and salts (Mexoryl®SX)
4-tert-butyl-4′-methoxydibenzoylmethane (Neo Heliopan®357)
3-(4′-sulpho)benzylidenebornan-2-one and salts
2-ethylhexyl 2-cyano-3,3-diphenylacrylate (Neo Heliopan®303)
N-[(2 and 4)-[2-(oxoborn-3-ylidene)methyl]benzyl]acrylamide polymer
2-ethylhexyl p-methoxycinnamate (Neo Heliopan®AV)
ethyl p-aminobenzoate (25 mol) ethoxylated
isoamyl p-methoxycinnamate (Neo Heliopan®E1000)
2,4,6-trianilino(p-carbo-2′-ethylhexyl-1′-oxy)-1,3,5-triazine (Uvinul®T150)
phenol, 2-(2H-benzotriazol-2-yl)-4-methyl-6-(2-methyl-3(1,3,3,3-tetramethyl-1-(trimethylsilyl)oxy)disiloxyanyl)propyl), (Mexoryl®XL)
4,4′-[(6-[4-(1,1-dimethyl)aminocarbonyl)phenylamino]-1,3,5-triazin-2,4-diyl)-diimino]bis(benzoic acid 2-ethylhexyl ester), (UvasorbHEB)
3-(4′-methylbenzylidene)-d,l-camphor (Neo Helipan®MBC)
3-benzylidenecamphor
2-ethylhexyl salicylate (Neo Helipan®OS)
2-ethylhexyl 4-dimethylaminobenzoate (Padimate O)
hydroxy-4-methoxybenzophenone-5-sulphonic acid and Na salt
2,2′-methylenebis(6-(2H-benzotriazol-2-yl)-4-1,1,3,3-tetramethylbutyl)-phenol), (Tinosorb®M)
phenylenebisbenzimidazyltetrasulphonic acid disodium salt (Neo Heliopan®AP)
2,4-bis[{(4-(2-ethylhexyloxy)-2-hydroxy}phenyl]-6-(4-methoxyphenyl)-1,3,5-triazine, (Tinosorb®S)
benzylidenemalonate-polysiloxane (Parsol®SLX)
menthyl anthranilate (Neo Heliopan®MA)
hexyl 2-(4-diethylamino-2-hydroxybenzoyl)benzoate (Uvinul® A Plus)
indanylidene compounds as per DE 100 55 940 (=WO 02/38537).

It is possible, furthermore, to use particulate UV filters or inorganic pigments, which if desired may have been rendered hydrophobic, such as the oxides of titanium (TiO₂), of zinc (ZnO), of iron (Fe₂O₃), of zirconium (ZrO₂), of silicon (SiO₂), of manganese (z.B. MnO), of aluminium (Al₂O₃), of cerium (e.g. Ce₂O₃) and/or mixtures.

Preferred embodiments of the cosmetic and/or pharmaceutical, especially dermatologically active, preparations of the invention generally include a fraction of (skin and/or hair) care substances in the range from 0.01% to 10% by weight, preferably in the range from 0.1% to 8% by weight. According to one preferred embodiment the compositions comprise one or more animal and/or vegetable care fats and oils (which are then part of the oil phase), such as olive oil, sunflower oil, refined soya oil, palm oil, sesame oil, rapeseed oil, almond oil, borage oil, evening primrose oil, coconut oil, shea butter, jojoba oil, sperm oil, beef tallow, neat's-foot oil and lard.

Preferred embodiments of the cosmetic and/or pharmaceutical, especially dermatologically active, preparations of the invention comprise, if desired, further ingredients having care properties, such as, for example, fatty alcohols having 6 to 30 C atoms. The fatty alcohols here can be saturated or unsaturated and linear or branched. Furthermore, these fatty alcohols can in some cases be part of the oil phase (III) if they correspond to the definition given there. Alcohols which can be employed are, for example, decanol, decenol, octanol, octenol, dodecanol, dodecenol, octadienol, decadienol, dodecadienol, oleyl alcohol, ricinoleyl alcohol, erucyl alcohol, stearyl alcohol, isostearyl alcohol, cetyl alcohol, lauryl alcohol, myristyl alcohol, arachidyl alcohol, caprylyl alcohol, capryl alcohol, linoleyl alcohol, linolenyl alcohol and behenyl alcohol, and also Guerbet alcohols thereof, such as, for example, 2-octyl-1-dodecanol, it being possible for the list to be extended virtually as desired by further alcohols of related structural chemistry. The fatty alcohols preferably originate from natural fatty acids, being conventionally prepared from the corresponding esters of the fatty acids by reduction. Fatty alcohol fractions which are formed by reduction from naturally occurring fats and fatty oils, such as beef tallow, peanut oil, colza oil, cottonseed oil, soya oil, sunflower oil, palm kernel oil, linseed oil, maize oil, castor oil, rapeseed oil, sesame oil, cacao butter and coconut fat, can further be employed.

Substances having care properties which can be employed in an outstanding manner in the cosmetic and/or dermatologically active preparations stabilized by means of the methods of the invention and comprising phenolic compounds of the formula (I), further include
ceramides, where ceramides are understood as meaning N-acylsphingosins (fatty acid amides of sphingosin) or synthetic analogues of such lipids (so-called pseudo-ceramides), which significantly improve the water retention capacity of the stratum corneum.
phospholipids, for example soya lecithin, egg lecithin and cephalins
fatty acids
phytosterols and phytosterol-containing fats or waxes
vaseline, paraffin oils and silicone oils; the latter include, inter alia, dialkyl- and alkylarylsiloxanes, such as dimethylpolysiloxane and methylphenylpolysiloxane, and also alkoxylated and quaternized derivatives thereof.

Animal and/or plant protein hydrolysates can advantageously also be added to preferred embodiments of cosmetic and/or pharmaceutical, especially dermatologically active, preparations of the invention. Substances which are advantageous in this respect are, in particular, elastin, collagen, keratin, milk protein, soya protein, oat protein, pea protein, almond protein and wheat protein fractions or corresponding protein hydrolysates, and also condensation products thereof with fatty acids, and quaternized protein hydrolysates, the use of plant protein hydrolysates being preferred.

Preferred embodiments of the cosmetic and/or pharmaceutical, especially dermatologically active, preparations of the invention may also comprise antioxidants, it being possible for all the antioxidants which are suitable or usual for cosmetic and/or dermatological applications to be used. The antioxidants are advantageously chosen from the group consisting of amino acids (e.g. glycine, histidine, tyrosine, tryptophan) and derivatives thereof, imidazoles (e.g urocanic acid) and derivatives thereof, peptides such as DL-carnosine, D-carnosine, L-carnosine and derivatives thereof (e.g. anserine), carotinoids, carotenes (e.g. alpha-carotene, beta-carotene, lycopene) and derivatives thereof, liponic acid and iderivatives thereof (e.g. dihydroliponic acid), aurothioglucose, propyl-thiouracil and other thiols (z.B. thioredoxin, glutathione, cysteine, cystine, cystamine and glycosyl, N-acetyl, methyl, ethyl, propyl, amyl, butyl and lauryl, palmitoyl, oleyl, gamma-linoleyl, cholesteryl and glyceryl esters thereof) as well as salts thereof, dilauryl thiodipropionate, distearyl thiodipropionate, thiodipropionic acid and derivatives thereof (esters, ethers, peptides, lipids, nucleotides, nucleosides and salts) as well as sulphoximine compounds (e.g. buthionine sulphoximine, homocysteine sulphoximine, buthionine sulphone, penta-, hexa-, heptathionine sulphoximine) in very low tolerated dosages, furthermore (metal) chelators, e.g. alpha-hydroxy fatty acids, palmitic acid, phytic acid, lactoferrin, alpha-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiqinone and ubiquinol and derivatives thereof, vitamin C and derivatives (e.g. ascorbyl palmitate, Mg ascorbyl phosphate, ascorbyl acetate), tocopherols and derivatives (e.g. vitamin E, vitamin E aceate), vitamin A and derivatives thereof (vitamin A palmitate) as well as coniferylbenzoate of benzoin resin, rutic acid and derivatives thereof, ferulic acid and derivatives thereof, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenium methionine), stilbenes and derivatives thereof (e.g. stilbene oxide, trans-stilbene oxide) and derivatives (salts, esters, ethers, sugars, nucleotides, nucleosides, peptides and lipids) of these active compounds mentioned.

Preferred embodiments of the cosmetic and/or pharmaceutical, especially dermatologically active, preparations of the invention may advantageously also comprise vitamins and vitamin precursors, it being possible for all the vitamins and vitamin precursors which are suitable or usual for cosmetic and/or dermatological applications to be used. Those worth mentioning here are, in particular, vitamins and vitamin precursors, such as tocopherols, vitamin A, niacin acid and niacinamide, further vitamins of the B complex, in particular biotin, and vitamin C and panthenol and derivatives thereof, in particular the esters and ethers of panthenol, and cationically derivatized panthenols, such as panthenol triacetate, panthenol monoethyl ether and the monoacetate thereof and cationic panthenol derivatives.

Preferred embodiments of the cosmetic and/or pharmaceutical, especially dermatologically active, preparations of the invention may also comprise active anti-inflammatory and/or redness- and/or itching-alleviating compounds (anti-irritants). All the active anti-inflammatory or redness- and/or itching-alleviating compounds which are suitable or usual for cosmetic and/or dermatological applications can be used here. Active anti-inflammatory and redness- and/or itching-alleviating compounds which are advantageously employed are steroidal anti-inflammatory substances of the corticosteroid type, such as hydrocortisone, dexamethasone, dexamethasone phosphate, methylprednisolone or cortisone, it being possible for the list to be extended by addition of further steroidal anti-inflammatories. Non-steroidal anti-inflammatories can also be employed. Those to be mentioned here by way of example are oxicams, such as piroxicam or tenoxicam; salicylates, such as aspirin, Disalcid, Solprin or fendosal; acetic acid derivatives, such as diclofenac, fenclofenac, indomethacin, sulindac, tolmetin, or clindanac; fenamates, such as mefenamic, meclofenamic, flufenamic or niflumic; propionic acid derivatives, such as ibuprofen, naproxen, benoxaprofen or pyrazoles, such as phenylbutazone, oxyphenylbutazone, febrazone or azapropazone.

Alternatively, natural anti-inflammatory or redness- and/or itching-alleviating substances can be employed. Plant extracts, specific highly active plant extract fractions and highly pure active substances isolated from plant extracts can be employed. Extracts, fractions and active substances from camomile, aloe vera, Commiphora species, Rubia species, willow, rose-bay willow-herb, oats, and also pure substances, such as, inter alia, bisabolol, apigenin 7-glucoside, boswellic acid, phytosterols, glycyrrhizic acid, glabridin or licochalcone A, are particularly preferred. The preparations comprising compounds of the formula (I) can also comprise mixtures of two or more active anti-inflammatory compounds.

Bisabolol, boswellic acid, and also extracts and isolated highly pure active compounds from oats and Echinacea are particularly preferred for use in the context of the invention as anti-inflammatory and redness- and/or itching-alleviating substances, and alpha-bisabolol and extracts and isolated highly pure active compounds from oats are especially preferred.

The amount of anti-irritants (one or more compounds) in the preparations is preferably 0.0001% to 20% by weight, with particular preference 0.0001% to 10% by weight, in particular 0.001% to 5% by weight, based on the total weight of the preparation.

Preferred embodiments of the cosmetic and/or pharmaceutical, especially dermatologically active, preparations of the invention may advantageously also comprise moisture retention regulators. The following substances for example are used as moisture retention regulators (moisturizers): sodium lactate, urea, alcohols, sorbitol, glycerol, propylene glycol, aliphatic 1,2-diols with a C number of 5-10, collagen, elastin or hyaluronic acid, diacyl adipates, petrolatum, ectoin, urocanic acid, lecithin, pantheol, phytantriol, lycopene, algae extract, ceramides, cholesterol, glycolipids, chitosan, chondroitin sulphate, polyamino acids and polyamino sugars, lanolin, lanolin esters, amino acids, alpha-hydroxy acids (e.g. citric acid, lactic acid, malic acid) and derivatives thereof, sugars (e.g. inositol), alpha-hydroxy fatty acids, phytosterols, triterpene acids, such as betulinic acid or ursolic acid, algae extracts.

Preferred embodiments of the cosmetic and/or pharmaceutical, especially dermatologically active, preparations of the invention may advantageously also comprise mono-, di- and oligosaccharides, such as, for example, glucose, galactose, fructose, mannose, fruit sugars and lactose.

Preferred embodiments of the cosmetic and/or pharmaceutical, especially dermatologically active, preparations of the invention may advantageously also comprise plant extracts, which are conventionally prepared by extraction of the whole plant, but also in individual cases exclusively from blossom and/or leaves, wood, bark or roots of the plant. In respect of the plant extracts which can be used, reference is made in particular to the extracts which are listed in the table starting on page 44 of the 3rd edition of the Leiffaden zur Inhaltsstoffdeklaration kosmetischer Mittel [Manual of Declaration of the Constituents of Cosmetic Compositions], published by Industrieverband Korperpflegemittel und Waschmittel e.V. (IKW), Frankfurt. Extracts which are advantageous in particular are those from aloe, witch hazel, algae, oak bark, rose-bay willow-herb, stinging nettle, dead nettle, hops, camomile, yarrow, arnica, calendula, burdock root, horsetail, hawthorn, linden blossom, almond, pine needle, horse chestnut, sandalwood, juniper, coconut, mango, apricot, orange, lemon, lime, grapefruit, apple, green tea, grapefruit pip, wheat, oats, barley, sage, thyme, wild thyme, rosemary, birch, mallow, lady's smock, willow bark, restharrow, coltsfoot, hibiscus, ginseng and ginger root.

In this context, the extracts from aloe vera, camomile, algae, rosemary, calendula, ginseng, cucumber, sage, stinging nettle, linden blossom, arnica and witch hazel are particularly preferred. Mixtures of two or more plant extracts can also be employed. Extraction agents which can be used for the preparation of plant extracts mentioned are, inter alia, water, alcohols and mixtures thereof. In this context, among the alcohols lower alcohols, such as ethanol and isopropanol, but also polyhydric alcohols, such as ethylene glycol, propylene glycol and butylene glycol, are preferred, and in particular both as the sole extraction agent and in mixtures with water. The plant extracts can be employed both in pure and in diluted form.

Preferred embodiments of the cosmetic and/or pharmaceutical, especially dermatologically active, preparations of the invention may in numerous cases advantageously comprise the following preservatives:

Preservatives which are preferably chosen here are those such as benzoic acid, its esters and salts, propionic acid and its salts, salicylic acid and its salts, 2,4-hexadienoic acid (sorbic acid) and its salts, formaldehyde and paraformaldehyde, 2-hydroxybiphenyl ether and its salts, 2-zincsulphidopyridine N-oxide, inorganic sulphites and bisulphites, sodium iodate, chlorobutanolum, 4-ethylmercuryl(II)-5-amino-1,3-bis(2-hydroxybenzoic acid), its salts and esters, dehydracetic acid, formic acid, 1,6-bis(4-amidino-2-bromophenoxy)-n-hexane and its salts, the sodium salt of ethylmercury(II)-thiosalicylic acid, phenylmercury and its salts, 10-undecylenic acid and its salts, 5-amino-1,3-bis(2-ethylhexyl)-5-methyl-hexahydropyrimidine, 5-bromo-5-nitro-1,3-dioxane, 2-bromo-2-nitro-1,3-propanediol, 2,4-dichlorobenzyl alcohol, N-(4-chlorophenyl)-N′-(3,4-dichlorophenyl)urea, 4-chloro-m-cresol, 2,4,4′-trichloro-2′-hydroxydiphenyl ether, 4-chloro-3,5-dimethylphenol, 1,1′-methylene-bis(3-(1-hydroxymethyl-2,4-dioximidazolidin-5-yl)urea), poly(hexamethylene diguanide) hydrochloride, 2-phenoxyethanol, hexamethylenetetramine, 1-(3-chloroallyl)-3,5,7-triaza-1-azoniaadamantane chloride, 1-(4-chlorophenoxy)-1-(1H-imidazol-1-yl)-3,3-dimethyl-2-butanone, 1,3-bis-(hydroxymethyl)-5,5-dimethyl-2,4-imidazolidinedione, benzyl alcohol, octopirox, 1,2-dibromo-2,4-dicyanobutane, 2,2′-methylenebis(6-bromo-4-chlorophenol), bromochlorophene, mixture of 5-chloro-2-methyl-3(2H)-isothiazolinone and 2-methyl-3(2H)isothiazolinone with magnesium chloride and magnesium nitrate, 2-benzyl-4-chlorophenol, 2-chloroacetamide, chlorhexidine, chlorhexidine acetate, chlorhexidine gluconate, chlorhexidine hydrochloride, 1-phenoxypropan-2-ol, N-alkyl(C₁₂-C₂₂)trimethylammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxymethylurea, 1,6-bis(4-amidinophenoxy)-n-hexane and its salts, glutaraldehyde, 5-ethyl-1-aza-3,7-dioxabicyclo[3.3.0]octane, 3-(4-chlorophenoxy)-1,2-propanediol, hyamines, alkyl-(C₈-C₁₈)-dimethylbenzylammonium chloride, alkyl-(C₈-C₁₈)-dimethylbenzylammonium bromide, alkyl-(C₈-C₁₈)-dimethylbenzyl-ammonium saccharinate, benzyl hemiformal, 3-iodo-2-propynyl butylcarbamate, sodium hydroxymethylaminoacetate or sodium hydroxymethylaminoacetate.

In various cases it may also be advantageous to employ substances which are chiefly employed for inhibition of the growth of undesirable microorganisms on or in animal organisms in cosmetic and/or pharmaceutical, especially dermatologically active, preparations of the invention. In this respect, in addition to conventional preservatives, further active compounds which are worth mentioning, in addition to the large group of conventional antibiotics, are, in particular, the products relevant for cosmetics, such as triclosan, climbazol, octoxyglycerol, octopirox (1-hydroxy-4-methyl-6-(2,4,4-trimethylpentyl)-2(1H)-pyridone, 2-aminoethanol), chitosan, farnesol, glycerol monolaurate or combinations of the substances mentioned, which are employed, inter alia, against underarm odour, foot odour or dandruff formation.

Substances having a perspiration-inhibiting activity (antiperspirants) can moreover, be particularly advantageously employed in preferred embodiments of the cosmetic and/or pharmaceutical, especially dermatologically active, preparations of the invention for combating body odour. Perspiration-inhibiting active compounds which are employed are, above all, aluminium salts, such as aluminium chloride, aluminium chlorohydrate, nitrate, sulphate, acetate etc. In addition, however, the use of compounds of zinc, magnesium and zirconium may also be advantageous. For application in cosmetic and dermatological antiperspirants, the aluminium salts and—to a somewhat lesser extent—aluminium/zirconium salt combinations have substantially proved suitable. The aluminium hydroxychlorides which are partially neutralized and therefore tolerated better by the skin, but are not quite so active, are additionally worth mentioning. Alongside aluminium salts, further substances are also possible, such as, for example, a) protein-precipitating substances, such as, inter alia, formaldehyde, glutaraldehyde, natural and synthetic tannins and trichloroacetic acid, which bring about surface blockage of the sweat glands, b) local anaesthetics (inter alia dilute solutions of e.g. lidocaine, prilocalne or mixtures of such substances), which eliminate sympathetic supply of the sweat glands by blockade of the peripheral nerve pathways, c) zeolites of the X, A or Y type, which, alongside the reduction in secretion of perspiration, also function as absorbents for bad odours, and d) botulinus toxin (toxin of the bacterium Chlostridium botulinum), which is also employed in cases of hyperhidrosis, a pathologically increased secretion of perspiration, and the action of which is based on an irreversible blocking of the release of the transmitter substance acetylcholine, which is relevant for secretion of perspiration.

Furthermore, cosmetic and/or pharmaceutical, especially dermatologically active, preparations of the invention may also comprise substances having a cooling action. Individual active cooling compounds which are preferred for use in the context of the present invention are listed below. The skilled person is able to supplement the following list with a large number of further active cooling compounds; the active cooling compounds listed can also be employed in combination with one another: l-menthol, d-menthol, racemic menthol, menthone glycerol acetal (trade name: Frescolat®MGA), menthyl lactate (trade name: Frescolat®ML, menthyl lactate is preferably 1-menthyl lactate, in particular l-menthyl l-lactate), substituted menthyl-3-carboxylic acid amides (e.g. menthyl-3-carboxylic acid N-ethylamide), 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, N-acetylglycine menthyl ester, isopulegol, menthyl hydroxycarboxylic acid esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl 3,6-di- and -trioxaalkanoates, 3-menthyl methoxyacetate, icilin.

Preferred active cooling compounds are: l-menthol, d-menthol, racemic menthol, menthone glycerol acetal (trade name: Frescolat®MGA), menthyl lactate (preferably l-menthyl lactate, in particular l-menthyl l-lactate, trade name: Frescolat®ML), substituted menthyl-3-carboxylic acid amides (e.g. menthyl-3-carboxylic acid N-ethylamide), 2-isopropyl-N-2,3-trimethylbutanamide, substituted cyclohexanecarboxylic acid amides, 3-menthoxypropane-1,2-diol, 2-hydroxtethyl menthyl carbonate, 2-hydroxypropyl menthyl carbonate, isopulegol.

Particularly preferred active cooling compounds are: l-menthol, racemic menthol, menthone glycerol acetal (trade name: Frescolat®MGA), menthyl lactate (preferably l-menthyl lactate, in particular l-menthyl l-lactate, trade name: Frescolat®ML), 3-menthoxypropane-1,2-diol, 2-hydroxyethyl menthyl carbonate, 2-hydroxypropyl menthyl carbonate.

Very particularly preferred active cooling compounds are: l-menthol, menthone glycerol acetal (trade name: Frescolat®MGA), menthyl lactate (preferably l-menthyl lactate, in particular l-menthyl l-lactate, trade name: Frescolat®ML).

The use concentration of the active cooling compounds to be employed is, depending on the substance, preferably in the concentration range from 0.01% to 20% by weight, and more preferably in the concentration range from 0.1% to 5% by weight, based on the total weight of the completed (ready-to-use) cosmetic or pharmaceutical preparation.

Preferred embodiments of the cosmetic and/or pharmaceutical, especially dermatologically active, preparations of the invention may also comprise anionic, cationic, nonionic and/or amphoteric surfactants, especially if crystalline or microcrystalline solids, for example inorganic micropigments, are to be incorporated into the preparations. Surfactants are amphiphilic substances which can dissolve organic, nonpolar substances in water. According to the invention, surfactants therefore do not belong to the oil phase. In this context, the hydrophilic components of a surfactant molecule are usually polar functional groups, for example —COO⁻, —OSO₃²⁻, —SO₃⁻, while the hydrophobic parts as a rule are nonpolar hydrocarbon radicals. Surfactants are in general classified according to the nature and charge of the hydrophilic molecular moiety. A distinction can be made between four groups here:
anionic surfactants,
cationic surfactants,
amphoteric surfactants and
nonionic surfactants.

Anionic surfactants as a rule contain carboxylate, sulphate or sulphonate groups as functional groups. In aqueous solution, they form negatively charged organic ions in an acid or neutral medium. Cationic surfactants are almost exclusively characterized by the presence of a quaternary ammonium group. In aqueous solution, they form positively charged organic ions in an acid or neutral medium. Amphoteric surfactants contain both anionic and cationic groups and accordingly behave like anionic or cationic surfactants in aqueous solution, depending on the pH. In a strongly acid medium they have a positive charge, and in an alkaline medium a negative charge. On the other hand, they are zwitterionic in the neutral pH range. Polyether chains are typical of nonionic surfactants. Nonionic surfactants do not form ions in an aqueous medium.

### A. Anionic Surfactants

Anionic surfactants which are advantageously used are acylamino acids (and salts thereof), such as:
acyl glutamates, for example sodium acyl glutamate, di-TEA-palmitoyl aspartate and sodium caprylic/capric glutamate,
acyl peptides, for example palmitoyl hydrolysed milk protein, sodium cocoyl hydrolysed soya protein and sodium/potassium cocoyl hydrolysed collagen,
sarcosinates, for example myristoyl sarcosine, TEA-lauroyl sarcosinate, sodium lauroyl sarcosinate and sodium cocoyl sarcosinate,
taurates, for example sodium lauroyl taurate and sodium methylcocoyl taurate,
acyl lactylates, lauroyl lactylate, caproyl lactylate
alaninates
   carboxylic acids and derivatives, such as for example:
lauric acid, aluminium stearate, magnesium alkanolate and zinc undecylenate,
ester-carboxylic acids, for example calcium stearoyl lactylate, laureth-6 citrate and sodium PEG-4 lauramide carboxylate,
ether-carboxylic acids, for example sodium laureth-13 carboxylate and sodium PEG-6 cocamide carboxylate,
   phosphoric acid esters and salts, such as, for example, DEA-oleth-10 phosphate and dilaureth-4 phosphate,
   sulphonic acids and salts, such as
acyl isethionates, e.g. sodium/ammonium cocoyl isethionate,
alkylarylsulphonates,
alkylsulphonates, for example sodium coco-monoglyceride sulphate, sodium C12-14 olefinsulphonate, sodium lauryl sulphoacetate and magnesium PEG-3 cocamide sulphate,
sulphosuccinates, for example dioctyl sodium sulphosuccinate, disodium laureth-sulphosuccinate, disodium laurylsulphosuccinate and disodium undecylenamido-MEA-sulphosuccinate
   and
   sulphuric acid esters, such as:
alkyl ether sulphate, for example sodium, ammonium, magnesium, MIPA, TIPA laureth sulphate, sodium myreth sulphate and sodium C12-13 pareth sulphate,
alkyl sulphates, for example sodium, ammonium and TEA lauryl sulphate.

### B. Cationic Surfactants

Cationic surfactants which are advantageously used are
alkylamines,
alkylimidazoles,
ethoxylated amines,
quaternary surfactants,
RNH₂CH₂CH₂COO⁻ (at pH=7)
RNHCH₂CH₂COO— B⁺ (at pH=12) B⁺=any desired cation, e.g. Na⁺ and
ester quats.

Quaternary surfactants contain at least one N atom which is covalently bonded to 4 alkyl or aryl groups. This leads to a positive charge, independently of the pH. Alkylbetaine, alkylamidopropylbetaine and alkylamidopropylhydroxysulphaine are advantageous. The cationic surfactants used can further preferably be chosen from the group consisting of quaternary ammonium compounds, in particular benzyltrialkylammonium chlorides or bromides, such as, for example, benzyldimethylstearylammonium chloride, and also alkyltrialkylammonium salts, for example cetyltrimethylammonium chloride or bromide, alkyldimethyl-hydroxyethylammonium chlorides or bromides, dialkyldimethylammonium chlorides or bromides, alkylamideethyltrimethylammonium ether sulphates, alkylpyridinium salts, for example lauryl- or cetylpyridinium chloride, imidazoline derivatives and compounds having a cationic character, such as amine oxides, for example alkyldimethylamine oxides or alkylaminoethyldimethylamine oxides. Cetyltrimethylammonium salts in particular are advantageously used.

### C. Amphoteric Surfactants

Amphoteric surfactants which are advantageously to be used are
acyl/dialkylethylenediamine, for example sodium acylamphoacetate, disodium acylamphodipropionate, disodium alkylamphodiacetate, sodium acylamphohydroxypropylsulphonate, disodium acylamphodiacetate and sodium acylamphopropionate,
N-alkylamino acids, for example aminopropyl alkylglutamide, alkyl-aminopropionic acid, sodium alkylimidodipropionate and lauroampho-carboxyglycinate.

### D. Nonionic Surfactants

Nonionic surfactants which are advantageously used are
alcohols,
alkanolamides, such as cocamides MEA/DEA/MIPA,
amine oxides, such as cocoamidopropylamine oxide,
esters which are formed by esterification of carboxylic acids with ethylene oxide, glycerol, sorbitan or other alcohols,
ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glucoside and coco-glycoside.
sucrose esters, sucrose ethers
polyglycerol esters, diglycerol esters, monoglycerol esters
methylglucose esters, esters of hydroxy acids

The use of a combination of anionic and/or amphoteric surfactants with one or more nonionic surfactants is further advantageous.

In this context, the surface-active substance(s) can be present in a preparation of the invention in an amount in the range from 0.5% to 98% by weight, based on the total weight of the preparation.

Preferred embodiments and further aspects of the present invention emerge from the attached patent claims and the following examples. Unless stated otherwise, all data are based on weight.

### EXAMPLES OF METHODS OF PRODUCING COLOUR-STABLE PREPARATIONS COMPRISING COSMETIC AND/OR DERMATOLOGICALLY ACTIVE PHENOLIC COMPOUNDS OF THE FORMULA (I)

The examples below set out methods which are particularly effective in accordance with the invention for stabilizing cosmetic formulations. An improvement to the stability of phenolic compounds of the formula (I) in cosmetic and pharmaceutical preparations is achieved in this context by various methods of the invention, in particular through the inventive addition of photoprotective filters of the formula (II), but also through the addition of metal chelators and/or through adjustment of the pH to values of less than or equal to 5.5 and preferably less than or equal to 4.5, and also through the combination of the stated methods.

### Example 1

### Preparation of a Colour-Stable Oil Phase for Incorporation into Cosmetic and Pharmaceutical Preparations Containing 4-(1-phenylethyl)-1,3-dihydroxybenzene (CARN: 85-27-8; Formula (IA)), the Compound of the Formula (IA) having been Predissolved, for the Purpose of Colour Stabilization, in an Oil Phase (2-ethylhexyl Isononanoate; INCI Ethylhexyl Isononanoate) Additionally Containing 2-hydroxy-4-methoxybenzophenone (INCI: Benzophenone-3, Formula (IIC))

Stability tests with 4-(1-phenylethyl)-1,3-dihydroxybenzene solutions (CARN: 85-27-8; compound of formula (IA)) have shown that on irradiation with light (light source: Ultra-Vitalux W300) the substance exhibits a certain instability, which is manifested in a yellowish to brownish discoloration. In order to examine to what extent the stability of the compound of the formula (IA) can be increased by predissolution in an oil phase to which an oil-soluble photoprotective filter of the formula (II) is added, irradiation experiments were conducted. For this purpose the solutions listed in Table 1 were prepared. The oil phase used was 2-ethylhexyl isononanoate (INCI: Ethylhexyl isononanoate). The amount of 4-(1-phenylethyl)-1,3-dihydroxybenzene (formula (IA) was in this case in a range from 10% to 20% by weight, based on the amount of oil phase. The amount of the photoprotective filter added for colour stabilization, 2-hydroxy-4-methoxybenzophenone (INCI: Benzophenone-3, formula (IIC)), was in each case 1% by weight. In order to examine to what extent the addition of a photoprotective filter of the formula (II) is advantageous, all of the samples were irradiated for 24 hours with light (light source: Ultra-Vitalux W300). For the measurement of any colour changes that occurred, all of the samples were subjected to chromametry both before and after irradiation to determine the b* values, which represent a measure of the shift in the blue-yellow axis and so give a direct indication of a yellow discoloration occurring. The values determined in this way are likewise listed in Table 1.

TABLE 1
Formulas and chromametrically determined b* values of different oil
phases containing 4-(1-phenylethyl)-1,3-dihydroxybenzene (CARN: 85-27-8;
formula (IA)) to which 2-hydroxy-4-methoxybenzophenone (INCI:
Benzophenonene-3; formula (IIC)) has been added for the purpose of colour
stabilization

| | | | | | |
|---|---|---|---|---|---|
| Raw material | INCI | A | B | C | D |

Formulas (A-D)

| | | | | | |
|---|---|---|---|---|---|
| 4-(1-Phenylethyl)-1,3-dihydroxybenzene | Phenylethylresorcinol | 10 | 15 | 20 | 15 |
| 2-Ethylhexyl isononanoate | Ethylhexyl Isononanoate | 89 | 84 | 79 | 85 |
| 2-Hydroxy-4-methoxybenzophenone | Benzophenone-3 | 1 | 1 | 1 | — |

Result

| | | | | |
|---|---|---|---|---|
| b* value before 24 h UV test | 6.2 | 8.3 | 10.7 | 5.4 |
| b* value after 24 h UV test | 10.3 | 15.5 | 10.6 | 34.0 |

Result: as the irradiation experiments show, it is possible to reduce the discoloration of the solutions containing a phenolic compound significantly through the addition of photoprotective filters of the formula (II) such as, for example, benzophenone-3 (formula (IIC)). The b* values of the solutions containing 10, 15 or 20 percent by weight of 4-(1-phenylethyl)-1,3-dihydroxybenzene (CARN: 85-27-8; formula (IA)) to which, for the purpose of stabilization, 1 percent by weight in each case of the photoprotective filter 2-hydroxy-4-methoxybenzophenone (INCI: Benzophenone-3, formula (IIC)) was added, were significantly lower, at 10.3 and 15.5 and 10.6, than the value of the reference specimen (b* value: 34.0) which was not stabilized with 2-hydroxy-4-methoxybenzophenone (INCI: Benzophenone-3, formula (IIC)). The results thus show unambiguously that with the aid of the method of the invention of predissolving 4-(1-phenylethyl)-1,3-dihydroxybenzene (CARN: 85-27-8; formula (IA)) in an oil phase and the addition of photoprotective filters of the formula (II) such as benzophenone-3 (formula (IIC)) for example, it is possible to a high degree to prevent the light-induced degradation and the associated yellow to brown discoloration of phenolic compounds of the formula (I).

### Example 2

### Effect of pH on the Colour Stability of Aqueous-Ethanolic Solutions Containing 0.5% by Weight of 4-(1-phenylethyl)-1,3-dihydroxybenzene (CARN: 85-27-8; Formula (IA))

In order to examine to what extent the degradation and the associated yellow to brown discoloration of 4-(1-phenylethyl)-1,3-dihydroxybenzene (CARN: 85-27-8; formula (IA)) in cosmetic and pharmaceutical formulations is also influenced by the pH value of the preparation, serial pH experiments were conducted, by dissolving 4-(1-phenylethyl)-1,3-dihydroxybenzene (CARN: 85-27-8; formula (IA)) in aqueous/ethanolic solutions which had been adjusted to different pH values (pH 4, 5, 7 and 9, Table 2) using buffer solutions. The amount of 4-(1-phenylethyl)-1,3-dihydroxybenzene (CARN: 85-27-8; formula (IA)) was in each case 0.5 percent by weight. All of the samples were stored in the dark for 28 days at room temperature. In order to measure any colour changes occurring, all samples were subjected to chromametric determination, both at the beginning of the experiment and at the end of a storage time of 28 days, of the b* values, which represent a measure of the shift in the blue-yellow axis and therefore give a direct indication of any yellow discoloration occurring. The values determined in this way are listed in Table 2.

TABLE 2
Formulas and chromametrically determined b* values of different
aqueous-ethanolic solutions containing 4-(1-phenylethyl)-1,3-dihydroxybenzene
(CARN: 85-27-8; formula (IA)) which were adjusted to different pH values using
buffer solutions

| | | | | | |
|---|---|---|---|---|---|
| Raw material | INCI | A | B | C | D |

Formulas (A-D)

| | | | | | |
|---|---|---|---|---|---|
| 4-(1-Phenylethyl)-1,3-dihydroxybenzene | Phenylethylresorcinol | 0.5 | 0.5 | 0.5 | 0.5 |
| Ethyl alcohol, pure | Ethanol | 20.0 | 20.0 | 20.0 | 20.0 |
| Buffer solution pH 4.0 | Water | 79.5 | — | — | — |
| Buffer solution pH 5.0 | Water | — | 79.5 | | — |
| Buffer solution pH 7.0 | Water | — | — | 79.5 | — |
| Buffer solution pH 9.0 | Water | — | — | — | 79.5 |

Result

| | | | | |
|---|---|---|---|---|
| b* value, beginning of test | 0.5 | 0.7 | 2.8 | 10.2 |
| b* value after 28 days | 2.0 | 4.6 | 19.4 | 55.3 |

Result: as the pH value experimental series show, it is possible, by adjusting the pH value of ethanolic/aqueous solutions containing 0.5% of 4-(1-phenylethyl)-1,3-dihydroxybenzene (CARN: 85-27-8; formula (IA)) to values of less than or equal to 5.5, and in particular by adjusting the pH value to values of less than or equal to pH 4.5, to reduce significantly the discoloration of the solutions. A particularly strong discoloration could be found only for the solutions with a pH value of 9 (b*=55.3) and pH 7 (b*=19.4), whereas at pH 5 (b*=4.6) and pH 4 (b*=2.0) the only discoloration found was at a level which can be estimated as being minor (Table 2). The results therefore unambiguously show that by means of the method of the invention of adjusting the pH to values of less than or equal to 5.5 and preferably to values of less than or equal to 4.5 it is possible to a high degree to prevent the degradation and the associated discoloration of phenolic compounds of the formula (I).

### Example 3

### Effect of Adding Disodium Ethylenediaminetetraacetate (INCI: Disodium EDTA) on the Colour Stability of Ethanolic-Aqueous Solutions with Different pH Values Containing 0.5% of 4-(1-phenylethyl)-1,3-dihydroxybenzene (CARN: 85-27-8; Formula (IA)).

In order, further, to examine to what extent the stability of 4-(1-phenylethyl)-1,3-dihydroxybenzene (CARN: 85-27-8; formula (IA)) can be further improved through the addition of metal chelators to solutions with different pH values, the solutions of Example 2, which had been adjusted using buffer solutions to the pH values of 9, 7, 5 and 4, were each admixed with 0.15% by weight of the metal chelator disodium ethylenediaminetetraacetate (INCI: Disodium EDTA). The amount of 4-(1-phenylethyl)-1,3-dihydroxybenzene (CARN: 85-27-8; formula (IA)) was again in each case 0.5 percent by weight. The samples were likewise stored in the dark at room temperature for 28 days. In order to measure any colour changes occurring, again, all of the samples were subjected to chromometric determination of the b* values, which represent a measure of the shift in the blue-yellow axis and hence a direct indication of any yellow discoloration occurring; these measurements were made both at the beginning of the experiment and at the end of a storage period of 28 days. The values determined in this way are listed in Table 3.

TABLE 3
Formulas and chromametrically determined b* values of different
aqueous-ethanolic solutions containing 4-(1-phenylethyl)-1,3-dihydroxybenzene
(CARN: 85-27-8; formula (IA)) which were adjusted to different pH values using
buffer solutions and to which disodium ethylenediaminetetraacetate (INCI:
Disodium EDTA) was added for the purpose of further improving stabilization.

| | | | | | |
|---|---|---|---|---|---|
| Raw material | INCI | A | B | C | D |

Formulas (A-D)

| | | | | | |
|---|---|---|---|---|---|
| 4-(1-Phenylethyl)-1,3-dihydroxybenzene | Phenylethylresorcinol | 0.50 | 0.50 | 0.50 | 0.50 |
| Ethyl alcohol, pure | Ethanol | 20.0 | 20.0 | 20.0 | 20.0 |
| Buffer solution pH 4.0 | Water | 79.35 | — | — | — |
| Buffer solution pH 5.0 | Water | — | 79.35 | | — |
| Buffer solution pH 7.0 | Water | — | — | 79.35 | — |
| Buffer solution pH 9.0 | Water | — | — | — | 79.35 |
| Disodium ethylenediaminetetraacetate | Disodium EDTA | 0.15 | 0.15 | 0.15 | 0.15 |

Result

| | | | | |
|---|---|---|---|---|
| b* value, beginning of test | 0.1 | 0.3 | 1.8 | 6.9 |
| b* value after 28 days | 1.5 | 3.6 | 14.8 | 40.2 |

Results: As the experimental series outlined in Example 3 show, the addition of disodium ethylenediaminetetraacetate to ethanolic/aqueous solutions containing 0.5% of 4-(1-phenylethyl)-1,3-dihydroxybenzene (CARN: 85-27-8; formula (IA)), adjusted to values of less than or equal to pH 5.5 and preferably to pH values of less than or equal to pH 4.5 allows the discoloration of the solutions to be prevented even more effectively, so that the only discoloration found was at a level assessable as particularly minor (Table 3). At a pH of 5 (b*=4.6 without addition of disodium ethylenediaminetetraacetate) it was possible to reduce the b* value further to 3.6 through the addition of disodium ethylenediaminetetraacetate. At a pH of 4 (b*=2.0 without addition of disodium ethylenediaminetetraacetate) it was possible to reduce the b* value to b*=1.5 by adding disodium ethylenediaminetetraacetate. Conversely, the particularly severe discoloration associated with the solutions having a pH of 9 (b*=40.2) and a pH of 7 (b*=14.8) could not be adequately prevented even by the addition of disodium ethylenediaminetetraacetate. The results clearly show, however, that by means of the combination of the method of the invention of adjusting the pH to values of less than or equal to 5.5 and with particular preference to values of less than or equal to 4.5, in accordance with Example 2, and of the method of adding disodium ethylenediaminetetraacetate in accordance with Example 3, it is possible, to an additionally increased degree, to prevent the degradation and the associated discoloration of phenolic compounds of the formula (I), in comparison to Example 2 (pH value adjustment only).

### Example 4

### Production of Cosmetic and Pharmaceutical Preparations

Production of Particularly Colour-Stable Cosmetic and Pharmaceutical Preparations Containing 4-(1-phenylethyl)-1,3-dihydroxybenzene (CARN: 85-27-8; Formula (IA)), 4-(1-phenylethyl)-1,3-dihydroxybenzene (CARN: 85-27-8; Formula (IA)) having been Added, for the Purpose of Colour-Stabilizing the Preparation in Accordance with Example 1, with Predissolution in an Oil Phase (2-ethylhexyl Isononanoate; INCI Ethylhexyl Isononanoate) and the Preparation, Additionally, having been Additionally Stabilized both by Adjustment of the pH to Values of Less than or Equal to 5.5 and by the Addition of Disodium Ethylenediaminetetraacetate.

In order to examine to what extent the stability of 4-(1-phenylethyl)-1,3-dihydroxybenzene (CARN: 85-27-8; formula (IA)) in cosmetic and pharmaceutical preparations can be improved with particular advantage through the combination of the methods of Examples 1-3, preparations were produced to which 4-(1-phenylethyl)-1,3-dihydroxybenzene (CARN: 85-27-8; formula (IA)) had been added, either directly or with predissolution in an oil phase. Moreover, the preparations were adjusted by means of buffer solutions to a skin-physiologically acceptable pH value of 5.4. Furthermore, the metal chelator disodium ethylenediaminetetraacetate was added to the preparations. The amount of 4-(1-phenylethyl)-1,3-dihydroxybenzene (CARN: 85-27-8; formula (IA)) was again in each case 0.5 percent by weight. The preparations were likewise stored in the dark at room temperature for 28 days. For measurement of any colour changes occurring, again, all samples were subjected to chromametric determination of the b* values, which constitute a measure in the shift of the blue-yellow axis and hence give a direct indication of any yellow discoloration occurring; these measurements were made both at the beginning of the experiment and at the end of a storage period of 28 days. The precise compositions of the preparations, and the b* values determined as part of the stability tests, at the beginning and at the end of the storage period, are likewise listed in Table 4.

TABLE 4
Formulas and chromametrically determined b* values of different
cosmetic preparations containing 4-(1-phenylethyl)-1,3-dihydroxybenzene (CARN:
85-27-8; formula (IA)) and produced by different methods

| | | | | | |
|---|---|---|---|---|---|
| Raw material weight % | INCI | A | B | C | D |

Formulas (A-D)

| | | | | | |
|---|---|---|---|---|---|
| Neo PCL | Trideceth-9, | 1.5 | 1.5 | 1.5 | 1.5 |
| water-soluble N | PEG-5 Ethylhexanoate | | | | |
| Hydrolite-5 | Pentylene Glycol | 3.0 | 3.0 | 3.0 | 3.0 |
| EDTA BD | Disodium EDTA | — | 0.1 | 0.1 | — |
| Water, demineralized | Water (Aqua) | 86.3 | 85.7 | 83.7 | 86.8 |
| Dragocide liquid | Phenoxyethanol, Methyl, | 0.8 | 0.8 | 0.8 | 0.8 |
| | -Butyl, -Ethyl, Isobutyl, | | | | |
| | -Propylparaben | | | | |
| PCL liquid 100 | Cetearyl Octanoate | 3.0 | 3.0 | 3.0 | 3.0 |
| Stearic Acid | Stearic Acid | 2.0 | 2.0 | 2.0 | 2.0 |
| 4-(1-Phenylethyl)-1,3- | Phenylethylresorcinol | 0.5 | 0.5 | — | — |
| dihydroxybenzene | | | | | |
| 4-(1-Phenylethyl)-1,3- | 20% solution in | — | — | 2.5 | — |
| dihydroxybenzene | Dragoxat 89 | | | | |
| soln. | | | | | |
| Neo Heliopan BB | Benzophenone-3 | — | 0.5 | 0.5 | — |
| Paraffin oil | Mineral Oil | 2.0 | 2.0 | 2.0 | 2.0 |
| Abil 350 | Dimethicone | 0.3 | 0.3 | 0.3 | 0.3 |
| Pemulen TR1 | Acrylates C10-30 Alkyl | 0.25 | 0.25 | 0.25 | 0.25 |
| | Acrylate Crosspolymer | | | | |
| NAOH 10% sol. | Sodium Hydroxide | 0.35 | 0.35 | 0.35 | 0.35 |

| | | | | |
|---|---|---|---|---|
| Total | 100 | 100 | 100 | 100 |
| pH value: | 5.4 | 5.4 | 5.4 | 5.4 |

Result

| | | | | |
|---|---|---|---|---|
| b* value before 24 h UV test | 0.04 | 1.1 | 1.6 | −0.8 |
| b* value after 24 h UV test | 12.6 | 2.9 | 2.5 | −0.8 |

### Result:

As the stability investigations show, it is possible, through the combination of different methods, such as
the inventive predissolution of the phenolic compounds of formula (I) in an oil phase which, additionally, comprises inventive photoprotective filters of the formula (II)
   and
the additional adjustment of the pH to values of less than or equal to 5.5 and preferably less than or equal to 4.5
   and
   the addition of metal chelators, such as disodium ethylenediaminetetraacetate (INCI: Disodium EDTA),
   to reduce, in a particularly successful way, the discoloration of cosmetic and pharmaceutical preparations.

The lowest b* value of 2.5 and hence the lowest level of discoloration was achieved here for the preparation for which 4-(1-phenylethyl)-1,3-dihydroxybenzene (CARN: 85-27-8; formula (IA)) was added to the preparation in predissolved form in an oil phase containing Benzophenone-3, to which, additionally, disodium ethylenediaminetetraacetate was added, and whose pH value was adjusted to 5.4 (Table 4: column C: b*=2.5). The preparation which contained no Benzophenone-3 (formula (IIC)) and to which 1-phenylethyl-1,3-dihydroxybenzene (CARN: 85-27-8; formula (IA)) was also added not in predissolved form in an oil phase containing Benzophenone-3, and which additionally contained no disodium ethylenediaminetetraacetate, showed a substantially stronger discoloration after 28 days, as could be experimentally verified on the basis of the significantly higher b* value (see Table 4: column A; b*=12.6). The results therefore demonstrate without doubt that by means of the combination of the methods of Examples 1 to 3 it is possible to prevent almost completely the degradation and the associated discoloration of phenolic compounds of the formula (I).

### Example 5

### Production of Colour-Stable Preparations

### Further Examples of Colour-Stable Preparations Produced in Accordance with the Methods of the Invention of Stabilizing Phenolic Compounds of the General Formula (I) are Described Below

Table 5 lists by way of example further colour-stable preparations containing diphenols such as, for example, 4-(1-phenylethyl)-1,3-dihydroxybenzene (CARN: 85-27-8; formula (IA)) or, for example, 4-butylresorcinol (CARN: 18979-61-8). An improvement in the stability, and particularly the colour stability, is achieved through the addition of photoprotective filters of the formula (II), and in particular through the inventive addition of the phenolic compounds of the general formula (I) in a predissolved form in an oil phase additionally containing inventive photoprotective filters of the formula (II), it also being possible, in addition, to obtain a further prevention of degradation and of the associated colour changes through the addition of metal chelators and through the adjustment of the pH to values of less than or equal to 5.5 and preferably less than or equal to 4.5.

TABLE 5

| RAW MATERIAL | | | | | | | | | | |
|---|---|---|---|---|---|---|---|---|---|---|
| NAME | | | | | | | | | | |
| (MANUFACTURER) | INCI | 1 | 2 | 3 | 4 | 5 | 6 | 7 | 8 | 9 |
| Skin lightener or | | | | | | | | | | |
| mixture containing | | | | | | | | | | |
| skin lightener | | | | | | | | | | |
| 4-(1-Phenylethyl)1,3- | Phenylethylresorcinol | 10.0 | 5.0 | 1.0 | 5.0 | 2.5 | 0.5 | 2.5 | 1.0 | 2.5 |
| dihydroxybenzene and | and Ethylhexyl | | | | | | | | | |
| Dragoxat 89 and | Isononanoate and | | | | | | | | | |
| Neo Heliopan BB | Benzophenone-3 | | | | | | | | | |
| Ratio 20:79:1 | | | | | | | | | | |
| 4-Butylresorcinol and | 4-Butylresorcinol | | 0.25 | | | | | | 5.0 | |
| Dragoxat 89 and | and Ethylhexyl | | | | | | | | | |
| Neo Heliopan BB | Isononanoate and | | | | | | | | | |
| Ratio 20:79:1 | Benzophenone-3 | | | | | | | | | |
| β-Arbutin | Arbutin | | | | 0.5 | | | | 0.2 | |
| Kojic acid | Kojic Acid | | 0.5 | | | | | | | 1.0 |
| Mg ascorbyl- | Magnesium Ascorbyl- | | | | | | | | 3.0 | |
| phosphate | phosphate | | | | | | | | | |
| Liquorice extract | | | | | | | 2.0 | | | |
| Niacinamide | | | | 1.0 | | | | | | |
| Soja extract | | | 1.0 | | | | | | | 1.0 |
| Compounds of the | | | | | | | | | | |
| Formula (II) | | | | | | | | | | |
| Neo Heliopan ® BB | Benzophenone-3 | 0.5 | 0.5 | 0.5 | 0.5 | 0.5 | 0.5 | 0.5 | 0.5 | 0.5 |
| (Symrise) | | | | | | | | | | |
| Oil components | | | | | | | | | | |
| PCL Liquid 100 | Cetearyl Octanoate | 4.0 | 3.0 | | | | | 3.0 | | |
| Corapan TQ ® | Diethylhexyl 1,6- | | 2.0 | | | | | | | |
| (Symrise) | Naphthalate | | | | | | | | | |
| Dragoxat 89(Symrise) | Ethylhexyl Isononoate | | | 1.0 | 1.0 | | | | | |
| Isoadipate | Diisopropyl Adipate | | | | 1.0 | | | | | |
| Isopropyl myristate | Isopropyl Myristate | | | | | | | | | 4.0 |
| (Symrise) | | | | | | | | | | |
| Neutral oil (Symrise) | Caprylic/Capric/ | 4.0 | 2.0 | | | 2.0 | | | | |
| | Triglyceride | | | | | | | | | |
| Isodragol (Symrise) | Triisononanoin | | | | | | | | 6.0 | |
| Cetiol OE (Cognis) | Dicaprylyl Ether | | | | | | 3.0 | | | |
| Paraffin oil | Mineral Oil | | | | | | | | | 4.0 |
| Tegosoft TN ® | C12-C15 Alkylbenzoate | | | | 4.0 | 2.0 | | | | |
| (Goldschmidt) | | | | | | | | | | |
| Abil 100 ® | Dimethicone | 1.0 | 0.3 | | | | 1.0 | | 2.0 | 0.3 |
| (Goldschmidt) | | | | | | | | | | |
| Further ingredients | | | | | | | | | | |
| Bentone Gel M IO ® | Mineral Oil and | 3.0 | | | | | | | | |
| (Rheox) | Quaternium-18-hectorite | | | | | | | | | |
| | and Propylene Carbonate | | | | | | | | | |
| | Glyceryl Stearate and Cetyl | | | | | | | | | |
| | Alcohol | | | | | | | | | |
| alpha-Bisabolol | Bisabolol | | 0.1 | | 0.2 | 0.1 | 0.1 | | | |
| (Symrise) | | | | | | | | | | |
| 1,3-Butylene glycol | 1,3-Butylene Glycol | | 3.0 | | | | | | | |
| Carbopol 2050 ® | Carbomer | | 0.2 | | | 0.10 | | | | |
| (B.F. Goodrich) | | | | | | | | | | |
| Carbopol ETD 2001 | Carbomer | | | | 0.5 | | | | | |
| (Noveon) | | | | | | | | | | |
| Cetiol OE (Cognis) | Dicaprylyl Ether | | | | | | 3.0 | | | |
| Citric Acid | Citric Acid | | | | | | | 0.1 | 0.3 | |
| Copherol 1250 ® | Tocopherol Acetate | | 0.5 | | 0.5 | 0.5 | 0.5 | | | |
| (Cognis) | | | | | | | | | | |
| Dehyquart SP | Quaternium-52 | | | | | | | 0.5 | | |
| Dow Corning ® 193 | Dimethicone Polyol | | | 1.0 | | | | | | |
| (Dow corning) | | | | | | | | | | |
| D-Panthenol (BASF) | Panthenol | | | 0.5 | | | 0.5 | | | |
| Dracorin 100 s.e. | Glyceryl Stearate, PEG-100 | 3.0 | | | | | | | | |
| | Stearate | | | | | | | | | |
| Dragocid Liquid | Phenoxyethanol (and) | 0.8 | 0.8 | | 0.8 | 0.8 | 0.8 | 0.8 | 0.8 | |
| (Symrise) | Methylparaben (and) | | | | | | | | | |
| | Ethylparaben (and) | | | | | | | | | |
| | Butylparaben (and) | | | | | | | | | |
| | Propylparaben (and) | | | | | | | | | |
| | Isobutylparaben | | | | | | | | | |
| Dragophos S | Sodium Dihydroxycetyl | | | | | | | | 2.0 | |
| (Symrise) | Phosphate | | | | | | | | | |
| Dracorin CE | Glyceryl Stearate/Citrate | | | | | | | | 1.0 | |
| Dracorin GMS | Glyceryl Stearate | 2.0 | 2.0 | | | 2.0 | | | 2.0 | 3.0 |
| (Symrise) | | | | | | | | | | |
| Dracorin 100 s.e. P | Glyceryl Stearate, PEG-100 | | | | | | | | | 8.0 |
| (Symrise) | Stearate | | | | | | | | | |
| EDTA BD ® (BASF) | Disodium-EDTA | 0.15 | 0.15 | 0.15 | 0.15 | 0.15 | 0.15 | 0.15 | 0.15 | 0.15 |
| Emulgin B2 ® | Ceteareth-20 | 1.0 | | | | | | 0.7 | | |
| (Cognis) | | | | | | | | | | |
| Emulsiphos (Symrise) | Cetyl Phosphate, Hydrated | | 1.5 | | | 1.5 | | | | |
| | Palm Glycerides | | | | | | | | | |
| Ethanol (96%) | Ethyl Alcohol | | | 13.0 | 5.0 | | | | | |
| Extrapon Aloe Vera | | | | 1.0 | | | | | | |
| (Symrise) | | | | | | | | | | |
| Extrapon Kamille | | | | 1.0 | | | | | | |
| (Symrise) | | | | | | | | | | |
| Extrapon Hamamelis | | | | 1.0 | | | | | | |
| (Symrise) | | | | | | | | | | |
| Glycerin 99% | Glycerin | 3.0 | | 4.5 | | 3.0 | 4.0 | | | |
| Hydrolite-5 (Symrise) | Pentylene Glycol | | | | 4.5 | | 5.0 | | 3.0 | |
| Keltrol T ® (Calgon) | Xanthan Gum | | 0.2 | 0.2 | 0.3 | | | | | |
| Lanette E ® (Cognis) | Sodium Cetearyl Sulphate | | 0.7 | | | | | | | |
| Lanette O ® (Cognis) | Cetearyl Alcohol | 1.1 | | | | | | 2.5 | | |
| Lanette 16 ® (Cognis) | Cetyl Alcohol | | 1.2 | | | 0.5 | | | | 2.0 |
| Lanette 18 (Care | Stearyl Alcohol | | | | | | | | 4.5 | |
| Chemicals) | | | | | | | | | | |
| Lara Care A-200 | Galactoarabinan | | | | | | 0.2 | | | |
| (Rahn) | | | | | | | | | | |
| NaOH 10% aqueous | Sodium Hydroxide | | 2.8 | | 2.2 | 2.9 | 0.6 | | | 0.2 |
| solution | | | | | | | | | | |
| Natrosol 250 HHR | Hydroxymethyl Cellulose | 0.3 | | | | | | | | |
| (Aqualon) | | | | | | | | | | |
| Neo Heliopan ® AP | Disodium Phenyl- | | 22.0 | | | | | | | |
| (Symrise), 15% as | dibenzimidazoletetra- | | | | | | | | | |
| sodium salt | sulphonate | | | | | | | | | |
| Neo Heliopan ® AP | Disodium Phenyl- | | | 22.0 | | | | | | |
| (Symrise), (10% | dibenzimidazoletetra- | | | | | | | | | |
| aqueous solution | sulphonate | | | | | | | | | |
| neutralized with | | | | | | | | | | |
| NaOH) | | | | | | | | | | |
| Neo Heliopan ® AV | Ethylhexyl Methoxy- | | | | 5.0 | 6.0 | 2.0 | | | |
| (Symrise) | cinnamate | | | | | | | | | |
| Neo Heliopan ® 303 | Octocrylene | 7.0 | | | | | | | | |
| (Symrise) | | | | | | | | | | |
| Neo Heliopan ® 357 | Butyl Methoxydibenzoyl- | | 2.0 | | 1.5 | 1.5 | 1.5 | 0.5 | | |
| (Symrise) | methane | | | | | | | | | |
| Neo Heliopan ® | Isoamyl p-Methoxy- | | | | 5.0 | | 6.0 | 2.0 | | |
| E 1000 (Symrise) | cinnamate | | | | | | | | | |
| Neo Heliopan ® HMS | Homosalate | | 5.0 | | | | | | | |
| (Symrise) | | | | | | | | | | |
| Neo Heliopan ® | Phenylbenzimidazole- | | | 33.3 | 10.0 | 13.3 | | | | |
| Hydro (15% aqueous | sulphonic Acid | | | | | | | | | |
| solution neutralized | | | | | | | | | | |
| with NaOH) | | | | | | | | | | |
| (Symrise) | | | | | | | | | | |
| Neo Heliopan ® MA | Menthyl Anthranilate | 3.0 | | | | | | | | |
| (Symrise) | | | | | | | | | | |
| Neo Heliopan ® MBC | 4-Methylbenzylidene- | | | | 2.0 | 4.0 | 3.0 | | | |
| (Symrise) | camphor | | | | | | | | | |
| Neo Heliopan ® OS | Ethylhexyl Salicylate | 1.0 | | | | | | | | |
| (Symrise) | | | | | | | | | | |
| Neo PCL wssl. N | Trideceth-9, PEG-5 | | | 1.0 | 1.5 | | | | | |
| | Ethylhexanoate | | | | | | | | | |
| Perfume oil | Parfum (Fragrance) | 0.3 | 0.3 | 0.3 | 0.3 | 0.4 | 0.2 | 0.4 | 0.3 | 0.3 |
| Pemulen TR 2 | Acrylates/C10-30 Alkyl | | | | | | 0.2 | | | |
| (Novion) | Acrylate Crosspolymer | | | | | | | | | |
| 1,2-Propylene glycol | Propylene Glycol | | | | | | | | | 5.0 |
| Texapon N 70 | Sodium Laureth Sulphate | | | | | | 0.5 | | | |
| (Cognis) | | | | | | | | | | |
| Zinc Oxide neutral | Zinc Oxide | 5.0 | | | | | | | | |
| (Symrise) | | | | | | | | | | |
| Veegum ultra ® | Magnesium Aluminium | 1.0 | | | | | | | | |
| (Vanderbilt) | Sulphate | | | | | | | | | |
| Water, distilled | Aqua (Water) | ad | ad | ad | ad | ad | ad | ad | ad | ad |
| | | 100 | 100 | 100 | 100 | 100 | 100 | 100 | 100 | 100 |

| | | | | | | | | | |
|---|---|---|---|---|---|---|---|---|---|
| pH value | 4.0 | 5.4 | 4.3 | 5.4 | 5.4 | 5.2 | 5.0 | 4.9 | 4.8 |

Example 1: “Oil in water” emulsion with UVA/B broadband protection
Example 2: “Oil in water” emulsion with UVA/B broadband protection
Example 3: Sun spray with UVA/B broadband protection with low oil content
Example 4: Skin-lightening balm with UVA/UVB protection
Example 5: Skin-lightening aerosol foam with UVB/UVA protection
Example 6: Skin-lightening non-aerosol foam
Example 7: Skin-lightening hair conditioner with UVB/UVA protection
Example 8: Skin-lightening moisture cream O/W
Example 9: Skin-lightening face cream O/W

### SPECIFIC EMBODIMENTS

In specific embodiment one, the invention provides a cosmetic and/or pharmaceutical preparation comprising or consisting of
a) a tyrosinase-inhibiting amount of one or more compounds of the formula (I):

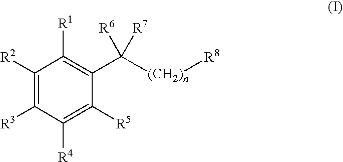

where for formula (I) it is the case that:
R¹, R², R³, R⁴ and R⁵ are independently of one another hydrogen, halogen, an OH group, methyl or a linear or branched alkyl having 2, 3, 4 or 5 C atoms, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl
   and
b) one or more compounds of the formula (II)

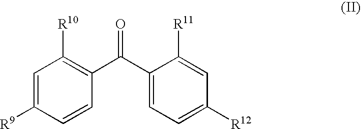

where for formula (II) it is the case that:
R⁹, R¹⁰, R¹¹ and R¹² independently of one another are hydrogen, methyl, an OH group or a group —O—R¹³
and
R¹³ is methyl or a linear or branched alkyl having 2, 3, 4, 5, 6, 7, 8, 9 or 10 C atoms.

2. In specific embodiment two, the invention provides a preparation according to specific embodiment one, where for one or more compounds of the formula (I) it is the case that:
R¹, R², R³, R⁴ and R⁵ are independently of one another hydrogen or an OH group, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl.

In specific embodiment three, the invention provides a preparation according to specific embodiment one, where for one or more compounds of the formula (I) it is the case that:
R¹, R², R³, R⁴ and R⁵ independently of one another are hydrogen, an OH group, halogen, methyl or a linear or branched alkyl having 2, 3 or 4 C atoms, of which two radicals from R¹, R², R³, R⁴ and R⁵ are hydrogen and two radicals are an OH group,
R⁶ is hydrogen, methyl or linear or branched alkyl having 2, 3, 4 or 5 C atoms,
R⁷ is methyl or a linear or branched alkyl having 2, 3, 4 or 5 C atoms,
R⁸ is a phenyl
and
n is 0.

In specific embodiment four, the invention provides a preparation according to any one of the preceding specific embodiments, comprising styrylresorcinol and/or 4-butylresorcinol as the compounds or one of the compounds of the formula (I).

In specific embodiment five, the invention provides preparation according to any one of the preceding specific embodiments, comprising or consisting of
(i) styrylresorcinol as compound of the formula (I)
   and
(ii) 2-hydroxy-4-methoxybenzophenone as compound of the formula (II)
in a weight ratio of 5:1.

In specific embodiment six, the invention provides a preparation according to any one of the preceding specific embodiment, wherein the preparation possesses a pH of less than or equal to 5.5, preferably less than or equal to 4.5.

In specific embodiment seven, the invention provides a preparation according to any one of the preceding specific embodiments, comprising one or more metal chelators.

In specific embodiment nine, the invention provides a preparation according to any one of the preceding specific embodiments, comprising water in an amount in the range from 25% to 95% by weight, preferably 40% to 90% by weight, based in each case on the total weight of the preparation.

In specific embodiment ten, the invention provides a preparation according to any one of the preceding specific embodiment, having an oil phase in an amount of 0.05% to 12% by weight, based on the total amount of the preparation.

In specific embodiment eleven, the invention provides a preparation according to specific embodiment 10 in the form of an O/W emulsion.

In specific embodiment twelve, the invention provides a preparation according to any one of the preceding specific embodiments, comprising one or more further UV filters and/or one or more further tyrosinase inhibitors.

In specific embodiment thirteen, the invention provides a preparation according to any one of the preceding specific embodiments, comprising a total amount of UV filters and/or inorganic pigments such that the preparation has a sun protection factor of greater than or equal to 2, preferably greater than or equal to 5.

In specific embodiment fourteen, the invention provides a preparation according to any one of the preceding specific embodiments, comprising a further active skin- and/or hair-lightening compound, preferably in an active skin- and/or hair-lightening amount.

In specific embodiment fifteen, the invention provides a preparation according to any one of the preceding specific embodiments, further comprising an active cooling compound in an amount sufficient to achieve a skin-cooling effect.

In specific embodiment sixteen, the invention provides a preparation according to any one of the preceding specific embodiments, further comprising one or more compounds for the care and/or cleansing of (a) skin and/or (b) hair.

In specific embodiment seventeen, the invention provides a preparation according to any one of the preceding specific embodiments, comprising a sensorially active amount of one or more odoriferous substances.

In specific embodiment eighteen, the invention provides a premix comprising or consisting of:
a) one or more compounds of the formula (I):

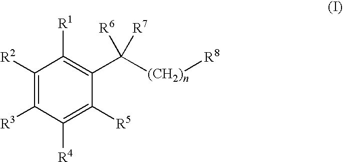

where for formula (I) it is the case that:
R¹, R², R³, R⁴ and R⁵ are independently of one another hydrogen, halogen, an OH group, methyl or a linear or branched alkyl having 2, 3, 4 or 5 C atoms, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl
   and
b) one or more compounds of the formula (II)

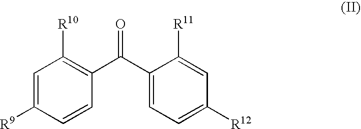

where for formula (II) it is the case that:
R⁹, R¹⁰, R¹¹ and R¹² independently of one another are hydrogen, methyl, an OH group or a group —O—R¹³
and
R¹³ is methyl or a linear or branched alkyl having 2, 3, 4, 5, 6, 7, 8, 9 or 10 C atoms,
   and
c) one or more oil components, the total amount of the oil component in the premix being 45% by weight or more, preferably 60% by weight or more, based on the total amount of the premix.

In specific embodiment nineteen, the invention provides a method of stabilizing compounds of the formula (I) by preparing a mixture comprising or consisting of:
a) one or more compounds of the formula (I)

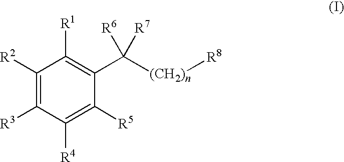

where for formula (I) it is the case that:
R¹, R², R³, R⁴ and R⁵ are independently of one another hydrogen, halogen, an OH group, methyl or a linear or branched alkyl having 2, 3, 4 or 5 C atoms, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl
   and
b) one or more compounds of the formula (II)

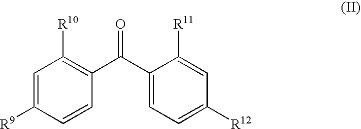

where for formula (II) it is the case that:
R⁹, R¹⁰, R¹¹ and R¹² independently of one another are hydrogen, methyl, an OH group or a group —O—R¹³
and
R¹³ is methyl or a linear or branched alkyl having 2, 3, 4, 5, 6, 7, 8, 9 or 10 C atoms
   and
c) if necessary one or more oil components.

In specific embodiment twenty, the invention provides a method according to specific embodiment nineteen, the resulting mixture being a preparation according to any one of specific embodiments one to seventeen or a premix according to specific embodiment eighteen.

In specific embodiment twenty-one, the invention provides a method according to specific embodiments nineteen or twenty, the resulting mixture being a preparation according to any one of specific embodiments one to seventeen, comprising or consisting of the following steps:
a) providing one or more compounds of the formula (I),
b) providing a mixture comprising one or more compounds of the formula (II) and one or more oil components, the total amount of the oil component being 45% by weight or more, based on the mixture,
c) dissolving one or more compounds of the formula (I) from step a) in the mixture from step b)
and subsequently
d) mixing the resulting premix from step c) with further ingredients of a cosmetic and/or pharmaceutical preparation.

In specific embodiment twenty-two, the invention provides a method of lightening skin and/or hair and/or of reducing age spots, comprising or consisting of the step of:
applying a preparation according to specific embodiments one to seventeen to skin and/or hair.

In specific embodiment twenty-three, the invention provides a use of one or more compounds of the formula (II)

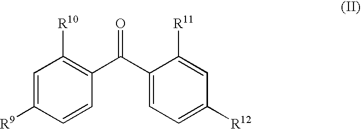

where for formula (II) it is the case that:
R⁹, R¹⁰, R¹¹ and R¹² are independently of one another hydrogen, methyl, an OH group or a group —O—R¹³
and
R¹³ is methyl or a linear or branched alkyl having 2, 3, 4, 5, 6, 7, 8, 9 or 10 C atoms,
for preparing a mixture comprising one or more compounds of the formula (II)
and
one or more compounds of the formula (I)

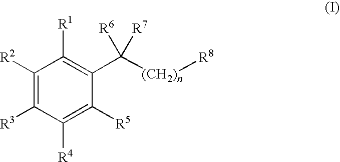

where for formula (I) it is the case that:
R¹, R², R³, R⁴ and R⁵ are independently of one another hydrogen, halogen, an OH group, methyl or a linear or branched alkyl having 2, 3, 4 or 5 C atoms, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl.

In specific embodiment twenty-four, the invention provides a use of one or more compounds of the formula (II)

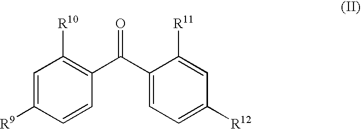

where for formula (II) it is the case that:
R⁹, R¹⁰, R¹¹ and R¹² are independently of one another hydrogen, methyl, an OH group or a group —O—R¹³
and
R¹³ is methyl or a linear or branched alkyl having 2, 3, 4, 5, 6, 7, 8, 9 or 10 C atoms,
for the long-term stabilization and/or colour stabilization of one or more compounds of the formula (I)

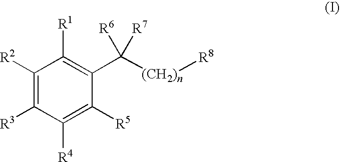

where for formula (I) it is the case that:
R¹, R², R³, R⁴ and R⁵ are independently of one another hydrogen, halogen, an OH group, methyl or a linear or branched alkyl having 2, 3, 4 or 5 C atoms, with 1, 2 or 3 different radicals from the group R¹, R², R³, R⁴ and R⁵ simultaneously representing an OH group,
R⁶ and R⁷ are independently of one another hydrogen, methyl or a linear or branched alkyl having 2, 3, 4 or 5 C atoms,
n is an integer from 0 to 20 and
R⁸ is methyl or a linear or branched alkyl having 2 or 3 C atoms, a cycloalkyl having 3, 4, 5, 6 or 7 C atoms or a phenyl.

It is claimed:

1. A cosmetic and/or pharmaceutical composition comprising
b) a tyrosinase-inhibiting amount of one or more compounds of the formula (I): 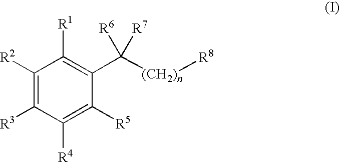
wherein:
R¹, R², R³, R⁴, and R⁵ are independently —H, halogen, —OH, methyl, or a linear or branched alkyl having 2, 3, 4, or 5 C atoms, wherein 1, 2, or 3 different moieties selected from the group consisting of R¹, R², R³, R⁴, and R⁵ simultaneously are —OH,
R⁶ and R⁷ are independently —H, methyl or a linear or branched alkyl having 2, 3, 4, or 5 C atoms,
n is an integer from 0 to 20, and
R⁸ is methyl or a linear or branched alkyl having 2 or 3 C atoms, cycloalkyl having 3, 4, 5, 6, or 7 C atoms, or phenyl,
and
b) one or more compounds of the formula (II) 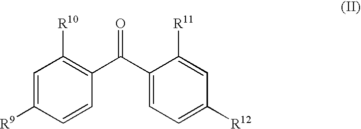
wherein:
R⁹, R¹⁰, R¹¹, and R¹² are independently-H, methyl, —OH or —O—R¹³
and
R¹³ is methyl or a linear or branched alkyl having 2, 3, 4, 5, 6, 7, 8, 9, or 10 C atoms.

2. The composition according to claim 1, wherein for one or more compounds of the formula (I):
R¹, R², R³, R⁴, and R⁵ are independently —H or —OH, wherein 1, 2, or 3 different moieties selected from the group consisting of R¹, R², R³, R⁴, and R⁵ are simultaneously —OH,
R⁶ and R⁷ are independently —H, methyl, or a linear or branched alkyl having 2, 3, 4, or 5 C atoms,
n is an integer from 0 to 20, and
R⁸ is methyl or a linear or branched alkyl having 2 or 3 C atoms, a cycloalkyl having 3, 4, 5, 6, or 7 C atoms, or phenyl.

3. The composition according to claim 1, wherein for one or more compounds of the formula (I):
R¹, R², R³, R⁴, and R⁵ are independently —H, —OH, halogen, methyl, or a linear or branched alkyl having 2, 3, or 4 C atoms, of which two moieties selected from the group consisting of R¹, R², R³, R⁴, and R⁵ are —H and two moieties are —OH,
R⁶ is —H, methyl, or linear or branched alkyl having 2, 3, 4, or 5 C atoms,
R⁷ is methyl or a linear or branched alkyl having 2, 3, 4, or 5 C atoms,
R⁸ is phenyl
and
n is 0.

4. The composition according to claim 1, comprising styrylresorcinol and/or 4-butylresorcinol as the compounds or one of the compounds of the formula (I).

5. The composition according to claim 1 comprising
(i) styrylresorcinol as the compound of formula (I)
and
(ii) 2-hydroxy-4-methoxybenzophenone as the compound of formula (II)
in a weight ratio of 5:1.

6. The composition according to claim 1, wherein the composition possesses a pH of less than or equal to 5.5.

7. The composition according to claim 1, further comprising one or more metal chelators.

8. The composition according to claim 1, further comprising water in an amount from 25% to 95% by weight based on the total weight of the composition.

9. The composition according to claim 1, further having an oil phase in an amount of 0.05% to 12% by weight, based on the total amount of the composition.

10. The composition according to claim 9, wherein the composition is an OQV emulsion.

11. The composition according to claim 1, further comprising one or more further UV filters and/or one or more further tyrosinase inhibitors.

12. The composition according to claim 1, comprising a total amount of UV filters and/or inorganic pigments such that the preparation has a sun protection factor of greater than or equal to 2.

13. The composition according to claim 1, further comprising a further active skin- and/or hair-lightening compound.

14. The composition according to claim 1, further comprising an active cooling compound in an amount sufficient to achieve a skin-cooling effect.

15. The composition according to claim 1, further comprising one or more compounds for the care and/or cleansing of (a) skin and/or (b) hair.

16. The composition according to claim 1, comprising a sensorially active amount of one or more odoriferous substances.

17. A premix composition comprising:
a) one or more compounds of the formula (I): 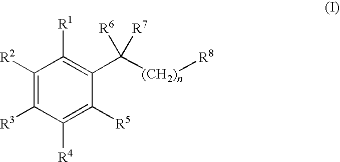
wherein:
R¹, R², R³, R⁴, and R⁵ are independently —H, halogen, —OH, methyl, or linear or branched alkyl having 2, 3, 4, or 5 C atoms, wherein 1, 2, or 3 different moieties selected from the group consisting of R¹, R², R³, R⁴, and R⁵ are simultaneously —OH,
R⁶ and R⁷ are independently —H, methyl, or a linear or branched alkyl having 2, 3, 4, or 5 C atoms,
n is an integer from 0 to 20, and
R⁸ is methyl or a linear or branched alkyl having 2 or 3 C atoms, cycloalkyl having 3, 4, 5, 6, or 7 C atoms, or phenyl
and
b) one or more compounds of the formula (II) 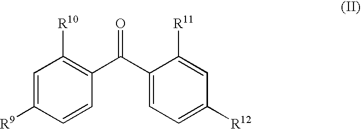
wherein:
R⁹, R¹⁰, R¹¹, and R¹² are independently —H, methyl, —OH, or —O—R¹³ and
R¹³ is methyl or a linear or branched alkyl having 2, 3, 4, 5, 6, 7, 8, 9, or 10 C atoms,
and
c) one or more oil components, wherein the total amount of the oil component in the premix composition is 45% by weight or more, based on the total amount of the premix composition.

18. A method for stabilizing compounds of the formula (I) comprising preparing a mixture comprising:
a) one or more compounds of the formula (I) 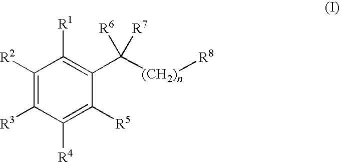
wherein
R¹, R², R³, R⁴, and R⁵ are independently —H, halogen, —OH, methyl, or a linear or branched alkyl having 2, 3, 4, or 5 C atoms, wherein 1, 2, or 3 different moieties selected from the group consisting of R¹, R², R³, R⁴, and R⁵ are simultaneously —OH,
R⁶ and R⁷ are independently —H, methyl, or a linear or branched alkyl having 2, 3, 4, or 5 C atoms,
n is an integer from 0 to 20, and
R⁸ is methyl or a linear or branched alkyl having 2 or 3 C atoms, cycloalkyl having 3, 4, 5, 6, or 7 C atoms, or phenyl
and
b) one or more compounds of the formula (II) 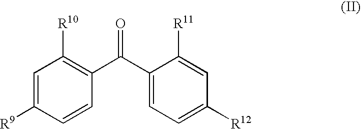
wherein:
R⁹, R¹⁰, R¹¹, and R¹² are independently —H, methyl, —OH, or —O—R¹³
and
R¹³ is methyl or a linear or branched alkyl having 2, 3, 4, 5, 6, 7, 8, 9, or 10 C atoms
and
c) optionally, one or more oil components.

19. A method for preparing the composition according to claim 1, comprising preparing a mixture comprising:
a) one or more compounds of the formula (I) 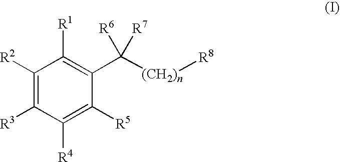
wherein
R¹, R², R³, R⁴, and R⁵ are independently-H, halogen, —OH, methyl, or a linear or branched alkyl having 2, 3, 4, or 5 C atoms, wherein 1, 2, or 3 different moieties selected from the group consisting of R¹, R², R³, R⁴, and R⁵ are simultaneously —OH,
R⁶ and R⁷ are independently-H, methyl, or a linear or branched alkyl having 2, 3, 4, or 5 C atoms,
n is an integer from 0 to 20, and
R⁸ is methyl or a linear or branched alkyl having 2 or 3 C atoms, cycloalkyl having 3, 4, 5, 6, or 7 C atoms, or phenyl
and
b) one or more compounds of the formula (II) 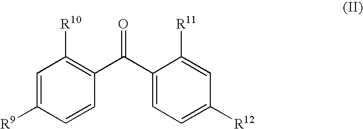
wherein:
R⁹, R¹⁰, R¹¹, and R¹² are independently-H, methyl, —OH, or —O—R¹³ and
R¹³ is methyl or a linear or branched alkyl having 2, 3, 4, 5, 6, 7, 8, 9, or 10 C atoms
and
c) optionally one or more oil components.

20. The method according to claim 19, further comprising:
a) providing one or more compounds of the formula (I),
b) providing a mixture comprising one or more compounds of the formula (II) and one or more oil components, the total amount of the oil component being 45% by weight or more, based on the mixture,
c) dissolving one or more compounds of the formula (I) from step a) in the mixture from step b) to form a premix,
and subsequently
d) mixing the premix from step c) with further ingredients of a cosmetic and/or pharmaceutical preparation.

21. A method of lightening skin and/or hair and/or of reducing age spots, comprising:
applying a composition according to claim 1 to skin and/or hair.

22. A method for preparing a formulation of one or more compounds of formula (II), comprising mixing one or more compounds of formula (II) 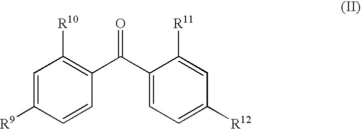
wherein:
R⁹, R¹⁰, R¹¹, and R¹² are independently —H, methyl, —OH or —O—R¹³ and
R¹³ is methyl or a linear or branched alkyl having 2, 3, 4, 5, 6, 7, 8, 9, or 10 C atoms,
and
one or more compounds of the formula (I) 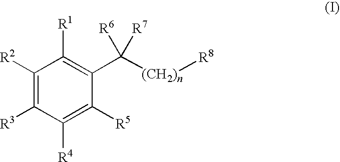
wherein:
R¹, R², R³, R⁴, and R⁵ are independently —H, halogen, —OH, methyl, or a linear or branched alkyl having 2, 3, 4, or 5 C atoms, wherein 1, 2, or 3 different moieties selected from the group consisting of R¹, R², R³, R⁴, and R⁵ are simultaneously —OH,
R⁶ and R⁷ are independently —H, methyl, or a linear or branched alkyl having 2, 3, 4, or 5 C atoms,
n is an integer from 0 to 20, and
R⁸ is methyl or a linear or branched alkyl having 2 or 3 C atoms, cycloalkyl having 3, 4, 5, 6, or 7 C atoms or phenyl.

23. A method for the long-term stabilization and/or colour stabilization of one or more compounds of the formula (I), comprising mixing one or more compounds of the formula (II) 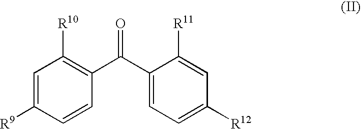
wherein:
R⁹, R¹⁰, R¹¹, and R¹² are independently —H, methyl, —OH or —O—R¹³
and
R¹³ is methyl or a linear or branched alkyl having 2, 3, 4, 5, 6, 7, 8, 9, or 10 C atoms,
and one or more compounds of the formula (I) 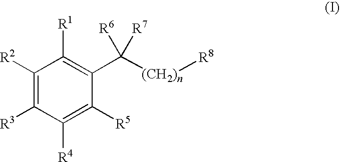
wherein:
R¹, R², R³, R⁴, and R⁵ are independently —H, halogen, —OH, methyl, or a linear or branched alkyl having 2, 3, 4, or 5 C atoms, wherein 1, 2, or 3 different moieties selected from the group consisting of R¹, R², R³, R⁴, and R⁵ are simultaneously —OH,
R⁶ and R⁷ are independently —H, methyl, or a linear or branched alkyl having 2, 3, 4, or 5 C atoms,
n is an integer from 0 to 20, and
R⁸ is methyl or a linear or branched alkyl having 2 or 3 C atoms, cycloalkyl having 3, 4, 5, 6, or 7 C atoms, or phenyl.
